(19) United States
(12) Patent Application Publication
(10) Pub. No.: US 2025/0108194 A1
(43) Pub. Date: Apr. 3, 2025

(54) SELF-EXPANDABLE DRUG TRANSFER DEVICE

(71) Applicant: Boston Scientific Scimed, Inc., Maple Grove, MN (US)

(72) Inventors: Javier Palomar-Moreno, Galway (IE); Michelle Hannon, Galway (IE); Jeffrey Madden, Galway (IE); John Kilcooley, Gort (IE)

(73) Assignee: Boston Scientific Scimed, Inc., Maple Grove, MN (US)

(21) Appl. No.: 18/898,973

(22) Filed: Sep. 27, 2024

Related U.S. Application Data
(60) Provisional application No. 63/541,043, filed on Sep. 28, 2023.

(51) Int. Cl. A61M 25/10

(52) U.S. Cl. CPC A61M 25/10; A61M 2025/105; A61M 2025/1088

(57) ABSTRACT

Drug delivery systems for delivering a therapeutic agent to a lumen while allowing for fluid perfusion. An illustrative drug delivery system may comprise an inner elongate shaft extending from a proximal end to a distal end and including a lumen extending from the proximal end to the distal end thereof and an outer elongate shaft extending from a proximal end to a distal end and including a lumen extending from the proximal end to the distal end thereof. The outer elongate shaft may be disposed over and axially movable relative to the inner elongate shaft. A self-expanding drug delivery device may include a plurality of longitudinally extending struts and may be movable between a radially collapsed configuration and a radially expanded configuration. A drug coating may be disposed on at least a portion of a radially outward surface of the longitudinally extending struts.

### CROSS-REFERENCE TO RELATED APPLICATIONS

This application claims the benefit of priority under 35 U.S.C. § 119 of U.S. Provisional Application No. 63/541,043, filed Sep. 28, 2023, the entire disclosure of which is hereby incorporated by reference.

### TECHNICAL FIELD

The present disclosure pertains to the manufacturing of medical devices and more particularly to self-expandable drug transfer devices which allow for a flow of fluid during drug transfer.

### BACKGROUND

A wide variety of medical devices have been developed for medical use, for example, intravascular and/or intracardiac use. Some of these devices include guidewires, catheters, balloons, stents, and the like. These devices are manufactured by any one of a variety of different manufacturing methods and may be used according to any one of a variety of methods. Some of these medical devices may include a therapeutic agent. Of the known medical devices and methods, each has certain advantages and disadvantages. There is an ongoing need to provide alternative medical devices as well as alternative methods for manufacturing and using medical devices. This may include alternative medical devices for delivering a therapeutic agent.

### SUMMARY

The present disclosure pertains to medical devices and more particularly to systems and methods delivering a therapeutic agent to a target location.

In a first example, a drug delivery system may comprise an inner elongate shaft extending from a proximal end to a distal end and including a lumen extending from the proximal end to the distal end thereof, an outer elongate shaft extending from a proximal end to a distal end and including a lumen extending from the proximal end to the distal end thereof, the outer elongate shaft disposed over and axially movable relative to the inner elongate shaft, a self-expanding drug delivery device extending from a proximal end to a distal end, the self-expanding drug delivery device movable between a radially collapsed configuration and a radially expanded configuration, the self-expanding drug delivery device including a plurality of longitudinally extending struts, and a drug coating disposed on at least a portion of a radially outward surface of the longitudinally extending struts.

Alternatively or additionally to any of the examples above, in another example, the plurality of longitudinally extending struts may each comprise a paddle and a proximally extending connection member, the proximally extending connection member may have a width less than a width of the paddle.

Alternatively or additionally to any of the examples above, in another example, the proximally extending connection member may be coupled to the inner elongate shaft.

Alternatively or additionally to any of the examples above, in another example, the plurality of longitudinally extending struts may each comprise a distally extending connection member, the distally extending connection member may have a width less than a width of the paddle.

Alternatively or additionally to any of the examples above, in another example, the distally extending connection member may be coupled to the inner elongate shaft adjacent the distal end thereof.

Alternatively or additionally to any of the examples above, in another example, the outer elongate shaft may comprise a distal holding section disposed adjacent the distal end of the outer elongate shaft. The distal holding section may be configured to maintain the self-expanding drug delivery device in a radially collapsed configuration.

Alternatively or additionally to any of the examples above, in another example, the distal holding section may have a cross-sectional dimension greater than a cross-sectional dimension of the outer elongate shaft proximal to the distal holding section.

Alternatively or additionally to any of the examples above, in another example, the distal holding section may define a cavity having a plurality of recesses sized and shaped to receive a portion of the longitudinally extending struts.

Alternatively or additionally to any of the examples above, in another example, the plurality of recesses may each include one or more longitudinally extending channels. The one or more longitudinally extending channels may extend along a lateral side of said recess.

Alternatively or additionally to any of the examples above, in another example, the plurality of longitudinally extending struts may comprise one or more rails configured to be received within the one or more longitudinally extending channels.

Alternatively or additionally to any of the examples above, in another example, the plurality of longitudinally extending struts may be spaced about a circumference of the inner elongate shaft.

Alternatively or additionally to any of the examples above, in another example, proximal retraction of the outer elongate shaft may be configured to allow the self-expanding drug delivery device to move from the radially collapsed configuration to the radially expanded configuration.

Alternatively or additionally to any of the examples above, in another example, the inner elongate shaft may further comprise a cap disposed adjacent to the distal end thereof.

Alternatively or additionally to any of the examples above, in another example, the cap may be configured to be disposed adjacent to the distal end of the outer elongate shaft when the self-expanding drug delivery device is in the radially collapsed configuration.

Alternatively or additionally to any of the examples above, in another example, the drug delivery system may further comprise a handle coupled to the proximal end of the inner elongate shaft and the proximal end of the outer elongate shaft. The outer elongate shaft may be configured to be axially displaced relative to the handle.

In another example, a drug delivery system may comprise an inner elongate shaft extending from a proximal end to a distal end and including a lumen extending from the proximal end to the distal end thereof, an outer elongate shaft extending from a proximal end to a distal end and including a lumen extending from the proximal end to the distal end thereof, the outer elongate shaft disposed over and axially movable relative to the inner elongate shaft, a self-expanding drug delivery device extending from a proximal end to a distal end, the self-expanding drug delivery device movable between a radially collapsed configuration and a radially expanded configuration, the self-expanding drug delivery device including a plurality of longitudinally extending struts, a distal holding section disposed adjacent the distal end of the outer elongate shaft, the distal holding section configured to constrain the self-expanding drug delivery device in the radially collapsed configuration when the self-expanding drug delivery device is disposed within a cavity of the distal holding section, and a drug coating disposed on at least a portion of a radially outward surface of the longitudinally extending struts. When the self-expanding drug delivery device is disposed within the cavity of the distal holding section, the radially outward surface of the longitudinally extending struts may be spaced a distance from an inner surface of the distal holding section.

Alternatively or additionally to any of the examples above, in another example, the cavity of the distal holding section may have a plurality of recesses sized and shaped to receive a portion of the longitudinally extending struts.

Alternatively or additionally to any of the examples above, in another example, the plurality of recesses may each include one or more longitudinally extending channels. The one or more longitudinally extending channels may extend along a lateral side of said recess.

Alternatively or additionally to any of the examples above, in another example, the plurality of longitudinally extending struts may comprise one or more rails configured to be received within the one or more longitudinally extending channels.

Alternatively or additionally to any of the examples above, in another example, the drug delivery system may further comprise a handle coupled to the proximal end of the inner elongate shaft and the proximal end of the outer elongate shaft. The outer elongate shaft may be configured to be axially displaced relative to the handle.

Alternatively or additionally to any of the examples above, in another example, proximal retraction of the outer elongate shaft may be configured to allow the self-expanding drug delivery device to move from the radially collapsed configuration to the radially expanded configuration.

In another example, a drug delivery system may comprise an inner elongate shaft extending from a proximal end to a distal end and including a lumen extending from the proximal end to the distal end thereof, an outer elongate shaft extending from a proximal end to a distal end and including a lumen extending from the proximal end to the distal end thereof, the outer elongate shaft disposed over and axially movable relative to the inner elongate shaft, a self-expanding drug delivery device extending from a proximal end to a distal end, the self-expanding drug delivery device movable between a radially collapsed configuration and a radially expanded configuration, the self-expanding drug delivery device including one or more interwoven struts defining a plurality of open cells, and a drug coating disposed on at least a portion of a radially outward surface of the interwoven struts.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic perspective view of an illustrative localized drug transfer or delivery system in partial cross-section;

FIG. 2 is an enlarged perspective view of a distal end region of the system of FIG. 1 with the outer elongate shaft in a proximally retracted configuration;

FIG. 3 is a perspective view of the distal end region of the system of FIG. 1 with the outer elongate shaft in a distally advanced, or delivery configuration;

FIG. 4 is an enlarged proximal end region of the delivery system of FIG. 1 in partial cross-section;

FIG. 5 is a perspective view of the distal end region of the system of FIG. 1 with the drug delivery device in a partially deployed configuration;

FIG. 6 is a schematic perspective view of a distal end region of another illustrative drug transfer system;

FIG. 7 is a schematic side view of a distal end region of another illustrative drug transfer system with the system in an expanded, deployed configuration;

FIG. 8 is a schematic side view of a distal end region of another illustrative drug transfer or delivery system in a deployed configuration;

FIG. 9 is a side view of a distal end region of the system of FIG. 8 with the outer elongate shaft in a distally advanced or delivery configuration; and

FIG. 10 is a side view of the distal end region of the system of FIG. 8 with the outer elongate shaft in a partially proximally retracted configuration.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The terms “therapeutic agents,” “drugs,” “bioactive agents,” “pharmaceuticals,” “pharmaceutically active agents,” “active pharmaceutical ingredient,” and other related terms may be used interchangeably herein and include genetic therapeutic agents, non-genetic therapeutic agents, proteomes and cells. Therapeutic agents may be used singly or in combination. A wide range of therapeutic agent loadings can be used in conjunction with the devices of the present invention, with the pharmaceutically effective amount being readily determined by those of ordinary skill in the art and ultimately depending, for example, upon the condition to be treated, the nature of the therapeutic agent itself, the tissue into which the dosage form is introduced, and so forth.

The following detailed description should be read with reference to the drawings in which similar elements in different drawings are numbered the same. The drawings, which are not necessarily to scale, depict illustrative embodiments and are not intended to limit the scope of the invention.

The body includes various passageways such as blood vessels and body lumens. These passageways sometimes become occluded by, for example, a tumor, excessive proliferation of cells, inflammation, plaque of diverse nature (e.g., lipidic, fibrotic, calcific), or the like. To widen an occluded body vessel, balloon catheters can be used, for example, in angioplasty. In some embodiments, a balloon catheter can include an inflatable and deflatable balloon carried by a long and narrow catheter body. The balloon can be initially folded around the catheter body to reduce the radial profile of the balloon catheter for easy insertion into the body. During use, the folded balloon can be delivered to a target location in the vessel, for example, a portion occluded by plaque, by threading the balloon catheter over a guide wire previously located in the vessel. The balloon is then inflated, for example, by introducing a fluid (such as a gas or a liquid) into the interior of the balloon. Inflating the balloon can radially expand the vessel so that the vessel can permit an increased rate of blood flow. After use, the balloon is typically deflated and withdrawn from the body. In some instances, it may be desirable to coat, layer, or otherwise apply an elutable drug or therapeutic agent to an outer surface of the balloon to deliver and/or administer the drug or therapeutic agent to a lumen wall when the balloon is expanded. However, balloons may block the flow of blood during the time the balloon is expanded and transferring the therapeutic agent. Some patients, for example those with cardiac insufficiency, may not tolerate a device blocking the flow of blood for the time period required to transfer the therapeutic agent to the vessel wall. About 11 to 18% of patients with coronary artery disease (CAD) needing complex percutaneous coronary intervention (PCI) may present with an impaired left ventricular function (e.g., reduced ejection fraction). These patients, among others, may not tolerate reduced or occluded blood flow due to an interventional procedure.

In the presence of CAD, without valvular disease, heart failure (HF) is most commonly caused by left ventricular (LV) systolic dysfunction. For coronary artery disease in patients with ischemic cardiomyopathy, there may be a benefit of revascularizing patients with coronary disease and left ventricular dysfunction using percutaneous coronary intervention (PCI) or coronary artery bypass grafting (CABG) compared to medical (pharmacological) treatment alone. In the clinical management of patients with heart failure, PCI and CABG may be considered as complimentary revascularization approaches. Registry data suggest a benefit of coronary artery bypass grafting over PCI in patients with reduced ejection fraction.

However, in patients with focal disease and comorbidities PCI is reasonable, especially if complete revascularization is possible. (Phillips, H R, O'Connor, C M, Rogers, J, Revascularization for heart failure, American Heart Journal, Volume 153, Issue 4, Supplement, 2007, Pages 65-73, ISSN 0002-8703, https://doi.org/10.1016/j.ahj.2007.01.026.). These patients may tolerate a drug coated balloon to treat the lesion better if a self-expanding drug transfer device allowing for blood perfusion is used. The cohort of patients with impaired LV function and CAD that are candidates for PCI may be approximately 11%-18%. Disclosed herein are drug-coated devices which allow for blood perfusion during procedures such as, but not limited to, percutaneous coronary intervention (PCI). Further, the devices and systems disclosed herein may transfer a therapeutic agent to a treatment location without leaving an implantable device. Implantable devices may cause inflammation and in-device restenosis in some patients. While the devices and systems are described with respect to PCI, it is contemplated that the devices and systems may be used in other regions of the body where it is desired to transfer a therapeutic agent. In one illustrative embodiment, the devices and systems disclosed herein may be used to deliver a cancer treatment.

Generally, the devices and systems disclosed herein may include a retractable elongate shaft which allows for different configurations of the device including but not limited to, a proximally retracted configuration (for drug transfer at a targeted anatomy) and a distally advanced position or delivery configuration (for delivery system tracking to targeted anatomy over a guide wire). A self-expandable drug delivery device can be collapsed and re-captured by the movable and/or retractable elongate shaft. The self-expanding drug delivery device may remain attached to the drug delivery system (e.g., catheter) at a proximal end of the drug delivery device. The drug delivery device may also use distal connection members (tethering) that may extend distally from a distal end of a paddle or self-expandable braid to the distal end of the drug delivery device (nose cone or atraumatic bumper tip). The self-expandable device can be deployed and re-captured by actuating a thumbwheel in different directions or by pulling/pushing wires. While the self-expandable device is deployed, a therapeutic agent is transferred to the targeted anatomy (localized drug delivery) for the duration of the time period when the device is deployed. The device deployment may not completely occlude the targeted anatomy (for example, when treating coronary arteries, blood perfusion is permitted).

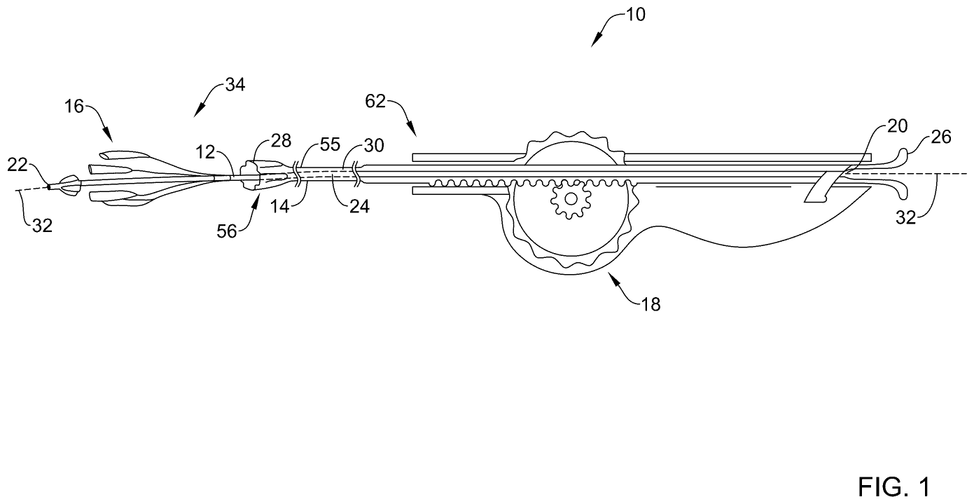

FIG. 1 is a schematic perspective view of an illustrative drug transfer or delivery system 10 in partial cross-section. The system 10 may generally include an inner elongate shaft 12, an outer elongate shaft 14, an expandable drug delivery device 16, and a handle 18. The inner elongate shaft 12 may extend from a proximal end 20 coupled to the handle 18 to a distal end 22. A lumen 24 may extend from the proximal end 20 to the distal end 22. While not explicitly shown, the lumen 24 of the inner elongate shaft 12 may be configured to receive a guidewire for navigation and tracking. In some examples, the lumen 24 may also be used to infuse diverse fluids, such as, but not limited to, saline solution, contrast media, or the like. The outer elongate shaft 14 may extend from a proximal end 26 movably coupled to the handle 18 to a distal end 28. A lumen 30 may extend from the proximal end 26 to the distal end 28. The inner elongate shaft 12 may be disposed within the lumen 30 of the outer elongate shaft 14. In some embodiments, the outer elongate shaft 14 may be configured to be axially displaced (e.g., proximally or distally) along a longitudinal axis 32 of the system 10 relative to the inner elongate shaft 12.

The cross-sectional dimensions of the inner elongate shaft 12 and/or the outer elongate shaft 14 may vary according to the desired application. Generally, the cross-sectional dimensions of the outer elongate shaft 14 may be sized smaller than the typical blood vessel in which the system 10 is to be used. The length of the outer elongate shaft 14 and/or the inner elongate shaft 12 may vary according to the location of the vascular passage where drug delivery is desired. In some instances, a 6 F or a 5 F catheter may be used as the outer elongate shaft 14, where “F,” also known as French catheter scale, is a unit to measure catheter diameter (1 F=⅓ millimeter (mm)). In some examples, intravascular ultrasound (IVUS) may be used for adequate sizing. Additionally, the inner elongate shaft 12 and/or the outer elongate shaft 14 or portions thereof may be selectively steerable. Mechanisms such as pull wires and/or other actuators may be used to selectively steer the inner elongate shaft 12 and/or the outer elongate shaft 14, if desired.

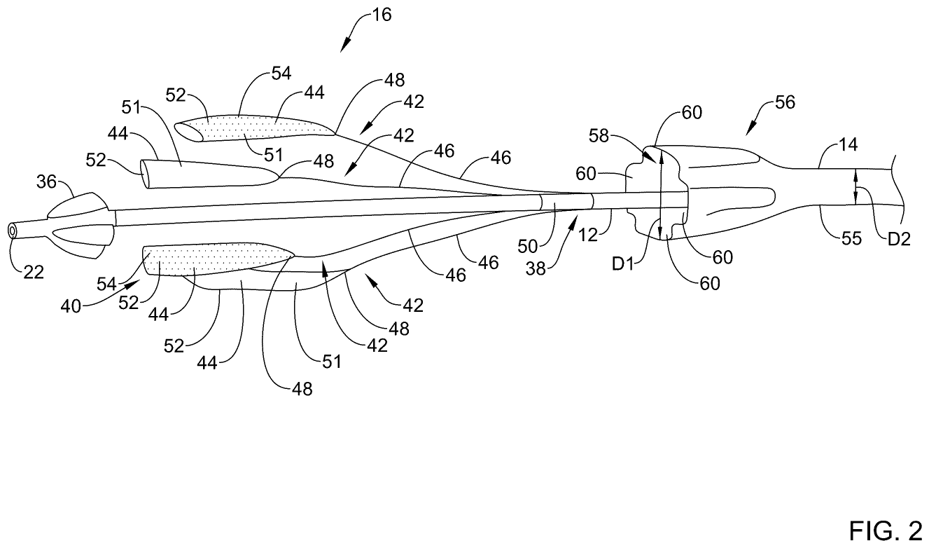

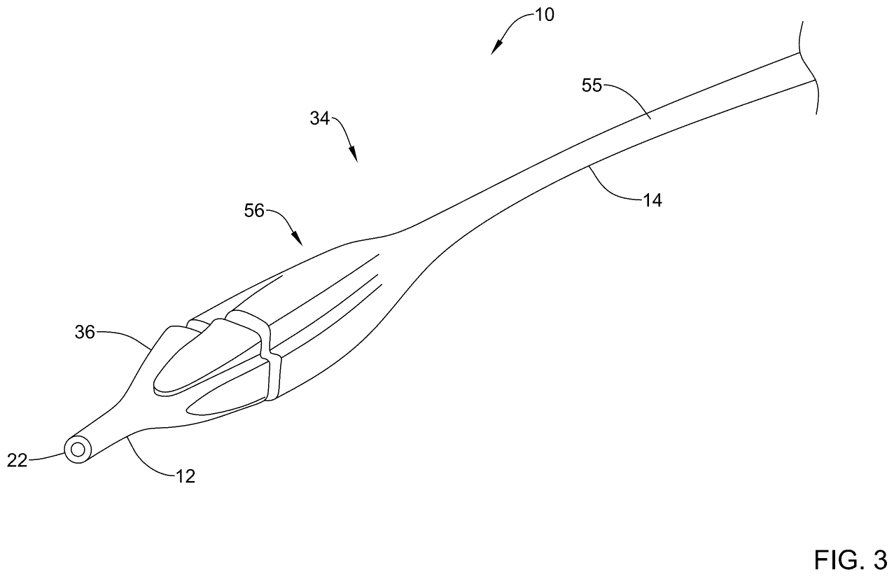

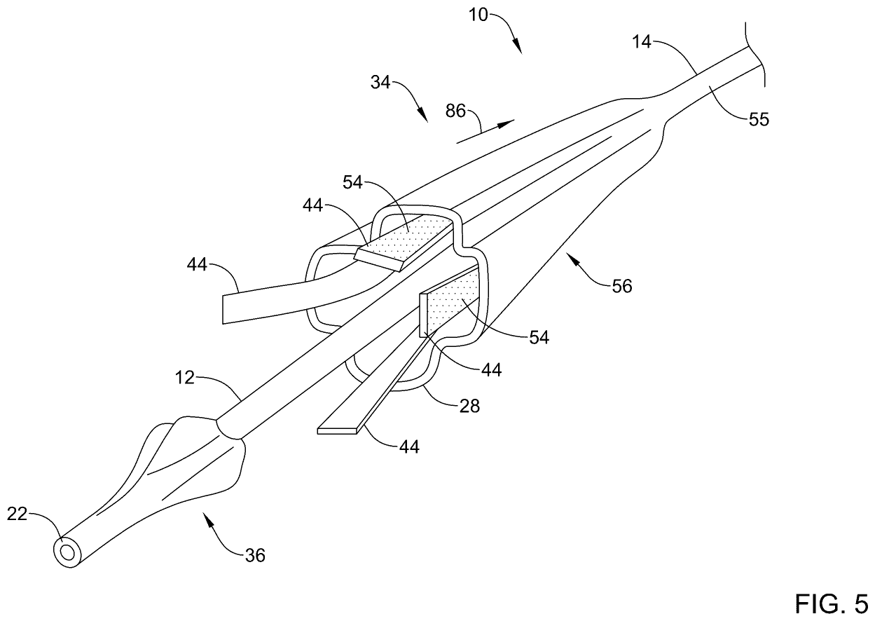

FIG. 2 is an enlarged perspective view of a distal end region 34 of the system 10 with the outer elongate shaft 14 in a proximally retracted configuration for drug transfer to a targeted anatomy. FIG. 3 is a perspective view of the distal end region 34 of the system 10 with the outer elongate shaft 14 in a distally advanced, or delivery configuration for tracking of the systems to a targeted anatomy over a guidewire. The inner elongate shaft 12 may include a nose cone, atraumatic bumper tip, or cap 36 positioned adjacent to the distal end 22 thereof. The cap 36 may have an outer diameter that is greater than an outer diameter of the distal end 22, although this is not required. The expandable drug delivery device 16 may be coupled to an outer surface of the inner elongate shaft 12. The expandable drug delivery device 16 may be configured to transition from a radially collapsed delivery configuration (see, for example, FIG. 5) to a radially expanded use configuration, as shown in FIG. 2. The expandable drug delivery device 16 may extend from a proximal end 38 to a distal end 40.

It is contemplated that the expandable drug delivery device 16 may be formed from a number of different materials such as, but not limited to, metals, metal alloys, shape memory alloys and/or polymers, as desired, enabling the expandable drug delivery device 16 to be biased into a radially compressed configuration for delivery and a radially expanded configuration when the expandable drug delivery device 16 is in use. Depending on the material selected for construction, the expandable drug delivery device 16 may be self-expanding (i.e., configured to automatically expand when unconstrained). As used herein the term “self-expanding” refers to the tendency of the expandable drug delivery device 16 to return to a preprogrammed shape when unrestrained from an external biasing force (for example, but not limited to, the outer elongate shaft 14, etc.). For example, the expandable drug delivery device 16 may be heat set into a radially expanded configuration, as shown in FIG. 2, and compressed into a radially collapsed configuration for delivery within the outer elongate shaft 14. As the expandable drug delivery device 16 exits the outer elongate shaft 14, the expandable drug delivery device 16 may resume the radially expanded configuration. In some embodiments, the expandable drug delivery device 16 may be formed from nitinol. However, this is not required. Other shape memory alloys and/or polymers may be used, as desired. It is further contemplated that the drug delivery device 16 may be manually expanded and/or collapsed through actuation of a pull wire or other actuation mechanism.

The expandable drug delivery device 16 may include a plurality of generally longitudinally extending struts 42. While the illustrated embodiment depicts four struts 42, it is contemplated that the system 10 may include fewer than four or more than four struts, as desired. The struts 42 may be uniformly spaced about a circumference of the inner elongate shaft 12, although this is not required. In some cases, the struts 42 may be eccentrically or non-uniformly spaced about the circumference of the inner elongate shaft 12. It is contemplated that the struts 42 may be circumferentially spaced from one another such that when the drug delivery device 16 is in a radially expanded configuration, the cross-section of the lumen in which drug delivery device 16 is deployed is not occluded. Said differently, when the drug delivery device 16 is in the radially expanded configuration blood or other fluid flows with no disruption or minimal disruption past the drug delivery device 16. Blood or fluid may flow through the spaces between the connection members 46.

Each strut 42 may include a paddle 44 positioned adjacent to the distal end 40 of the expandable drug delivery device 16. The paddles 44 may take any number of configurations desired. For example, the width of the paddles 44 may vary along a length thereof. In some cases, the width may taper or reduce from a distal end to a proximal end 48 of the paddle 44. Other shapes and/or configurations may be used as desired. For example, the paddles 44 may have a shape (when viewed from an outer side) that is generally rectangular, square, triangular, circular, oblong, polygonal, eccentric, or the like. A connection member 46 may extend proximally from the proximal end 48 of the paddle 44 to the proximal end 38 of the expandable drug delivery device 16. The connection members 46 may be secured to an outer surface of the inner elongate shaft 12 proximal to the distal end 22 thereof via a collar or other suitable coupling mechanism 50. A suitable securing method(s) may be employed to couple the collar 50 and the inner elongate shaft 12, including but not limited to adhesive bonding, thermal bonding (e.g., hot jaws, laser welding, etc.) or other bonding technique, as desired. In some examples, the connection members 46 may have a width or cross-sectional dimension that is less than a width of the paddles 44. For example, the connection members 46 may be sized and shaped to minimally impact the flow of blood when the expandable drug delivery device 16 is in the expanded configuration while securing the paddles 44 to the inner elongate shaft 12. In some examples, the connection members 46 may be formed from a wire. A distal end of the paddles 44 may be free from connection to the inner elongate shaft 12.

It is contemplated that the paddle 44 and the connection member 46 of each individual strut 42 may be formed as a single monolithic structure that are each individually coupled to the inner elongate shaft 12. In other examples, the paddle 44 and the connection member 46 of each individual strut 42 may be formed as separate components that are coupled to one another. In yet other examples, the expandable drug delivery device 16 may be formed as a single monolithic component. For example, the struts 42 and the collar 50 may be laser cut from a hypotube or may be 3-D printed.

The paddles 44 may include a radially inward surface 51. The radially inward surface 51 may be curved or concave to generally conform to an outer surface of the inner elongate shaft 12. It is contemplated that the paddles 44 may be curved to reduce a maximum outer diameter of the drug delivery device 16 in the collapsed delivery configuration. The paddles 44 may include a radially outward surface 52 having a drug coating 54 disposed thereon. In some examples, the radially outward surface 52 may have a generally convex shape configured to conform to an inner surface of a lumen. However, this is not required. The radially outward surface 52 of the paddles 44 may take any shape desired. The drug coating 54 may be disposed along substantially the entire length and/or width of each paddle 44 or along one or more portions of the paddles 44. Some paddles 44 may be free from the drug coating 54. The drug coating 54 disposed on the paddles 44 may have an average thickness in the range of about 1 micrometer (μm) to about 50 μm, for example, although the coating thickness may vary depending on the morphology of the therapeutic agent (e.g., crystalline), the treated anatomy, and/or the required drug dosage. In some embodiments, a light binder (not explicitly shown) may hold the drug coating 54 to the paddles 44. It is contemplated that the therapeutic agent may be grown on the outer surfaces 52 of the paddles 44.

In some embodiments, a flexible sleeve (not explicitly shown) may extend circumferentially between the paddles 44. The drug coating 54 may be applied to the outer surface of the flexible sleeve. It is contemplated that the flexible sleeve, if so provided, may have length less than a length of the drug delivery device 16. This may allow the surface area available to apply the drug coating 54 to be increased (relative to only using the outer surface 52 of the paddles 44) while allowing blood or other fluid to perfuse through the spaces between the connection members 46.

The drug coating 54 may include one or more therapeutic agents such as, but not limited to, anti-thrombotic agents, anti-proliferative agents, anti-inflammatory agents, direct oral anticoagulants (DOACs), anti-migratory agents, agents affecting extracellular matrix production and organization, antineoplastic agents, anti-mitotic agents, anesthetic agents, anti-coagulants, vascular cell growth promoters, vascular cell growth inhibitors, cholesterol-lowering agents, vasodilating agents, agents that interfere with endogenous vasoactive mechanisms, proteomes, and cytokines. More specific drugs or therapeutic agents include paclitaxel, rapamycin, sirolimus, everolimus, tacrolimus, heparin, diclofenac, aspirin, Epo D, dexamethasone, estradiol, halofuginone, cilostazol, geldanamycin, apixaban, rivaroxaban, edoxaban, dabigatran, betrixaban, argatroban, ABT-578 (Zotarolimus, Abbott Laboratories), trapidil, liprostin, actinomycin D, Resten-NG, Ap-17, abciximab, clopidogrel, Ridogrel, beta-blockers, bARKct inhibitors, phospholamban inhibitors, and SERCA 2 gene/protein, resiquimod, imiquimod (as well as other imidazoquinoline immune response modifiers), human apolipoproteins (e.g., AI, AII, AIII, AIV, AV, etc.), vascular endothelial growth factors (e.g., VEGF-2), antibody CD-34, interleukin-8, as well as derivatives of the forgoing, among many others, and/or combinations thereof.

In general, the drug delivery device 16 may be delivered to a suitable target region via the system 10 while in the collapsed configuration. Upon reaching the target region, the drug delivery device 16 may expand or be expanded into the expanded configuration. In the radially expanded configuration, the radially outward surfaces 52 of the paddles 44 may be configured to contact the lumen wall. The paddles 44 may be held in apposition with the lumen wall for a length of time sufficient to transfer the drug coating 54 from the paddles 44 to the lumen wall.

The outer elongate shaft 14 may be slidably disposed over the inner elongate shaft 12. The outer elongate shaft 14 may include a distal holding section 56 and a body portion 55. The body portion 55 may extend from the proximal end 26 of the outer elongate shaft 14 to a proximal end of the distal holding section 56. The distal holding section 56 may extend distally from a distal end of the body portion 55 to the distal end 28 of the outer elongate shaft 14. In some embodiments, the body portion 55 and the distal holding section 56 may be formed as single monolithic structure. In other embodiments, the body portion 55 and the distal holding section 56 may be formed as separate components that are subsequently coupled. A suitable securing method(s) may be employed to couple the body portion 55 and the distal holding section 56, including but not limited to adhesive bonding, thermal bonding (e.g., hot jaws, laser welding, etc.) or other bonding technique, as desired. The body portion 55 and/or the distal holding section 56 may be manufactured using a number of differing techniques, including, but not limited to, extrusion, metal additive 3-D printing, injection molding, and the like.

The distal holding section 56 may have a cross-sectional dimension D1 at the distal end 28 that is greater than a cross-sectional dimension D2 of at least a portion of the outer elongate shaft 14 proximal to the distal holding section 56. In some examples, the cross-sectional dimension of the distal holding section 56 may increase from the proximal end of the distal holding section 56 to the distal end 28 of the outer elongate shaft 14. The distal holding section 56 may be configured to receive the drug delivery device 16 therein. For example, an entire length of the drug delivery device 16 may be contained within the distal holding section 56 during delivery to a target site. In other examples, the paddles 44 of the drug delivery device 16 may be housed within the distal holding section 56 while the connection members 46 extend proximally into the body portion 55 of the outer elongate shaft 14. The distal holding section 56 may define a cavity 58 for slidably receiving the drug delivery device 16. The distal holding section 56 may have a cross-sectional shape configured to accommodate the drug delivery device 16. For example, in the illustrated embodiment, the distal holding section 56 may have a cross-sectional shape similar to a plus sign having rounded edges. Thus, the cavity 58 may have recesses 60 sized and shaped to the receive the paddles 44 therein. In some embodiments, there may be enough clearance (or spacing) between the radially outward surfaces 52 of the paddles 44 and he inner surface of the distal holding section 56 to prevent drug loss from friction between the movable outer elongate shaft 14 and the self-expanding drug delivery device 16. It is contemplated that the number of recesses 60 may be the same as a number of paddles 44. However, the distal holding section 56 may take other cross-sectional shapes, as desired, such as, but not limited to, circular, oblong, square, rectangular, polygonal, eccentric, and the like. As will be described in more detail herein the distal holding section 56 and/or the paddles 44 may include features configured to space the radially outward surfaces 52 of the paddles 44 from the inner surface of the distal holding section 56 when the outer elongate shaft 14 is disposed over the drug delivery device 16 to reduce or prevent contact between the drug coating 54 and the inner surface of the distal holding section 56.

The cap 36 of the inner elongate shaft 12 may be sized and shaped to mate with the distal end 28 of the outer elongate shaft 14 in the delivery configuration (see, for example, FIG. 3). This may enclose the drug delivery device 16 and prevent or reduce loss of the drug coating 54 as the drug delivery device 16 is navigated to the desired treatment location. The cap 36 may taper or reduce in cross-sectional dimension in the distal direction, although this is not required. It is contemplated that the cap 36 may have a cross-sectional shape similar to the cross-sectional shape of the distal holding section 56, although this is not required.

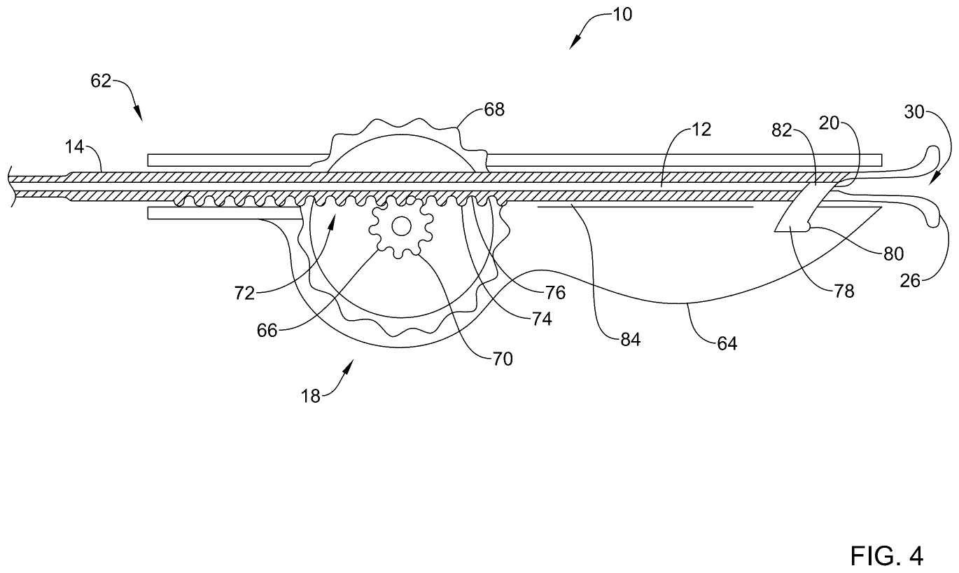

FIG. 4 illustrates an enlarged proximal end region 62 of the delivery system 10 in partial cross-section. For clarity, in FIG. 4, the inner elongate shaft 12 is not shown in cross-section. The proximal end region 62 of the drug delivery system 10 may include a handle 18 having a handle body 64 configured to remain outside the body. A lumen 84 may extend through the handle body 64. The outer elongate shaft 14 may be movably disposed within the lumen 84 of the handle 18. The handle body 64 may further include a thumbwheel 68. The thumbwheel 68 may be manipulated by an operator to drive a pinion 66. The pinion 66 may include gear teeth 70 configured to engage corresponding teeth and grooves 74, 76 on a rack 72 formed in an outer surface of the outer elongate shaft 14. Rotation of the thumbwheel 68 may rotate the pinion 66 causing longitudinal movement of the rack 72 and thus longitudinal movement of the outer elongate shaft 14 in the proximal or distal direction. It is contemplated that the outer elongate shaft 14 may be proximally retracted by rotating the thumbwheel 68 in a first direction and the outer elongate shaft 14 may be distally advanced by rotating the thumbwheel in a second direction opposite the first direction. While not explicitly shown, the handle 18 may include features such as a safety lock or a pawl to prevent or reduce unintentional movement or to limit the direction of movement of the rack 72.

The handle 18 may further include a bracket 78 configured to clamp to the proximal end region of the inner elongate shaft 12. The bracket 78 may extend from a first end 80 to a second end 82 configured to grip or clamp to the inner elongate shaft 12. The bracket 78 may be configured hold the inner elongate shaft 12 in a fixed orientation (e.g., longitudinally and rotationally) while the outer elongate shaft 14 is moved relative to the inner elongate shaft 12. In some examples, the first end 80 may extend to a location exterior to the handle body 64 to allow the bracket 78 to be released from the inner elongate shaft 12. However, this is not required.

FIG. 5 is a perspective view of the distal end region 34 of the system 10 with the drug delivery device 16 in a partially deployed configuration. It is contemplated that it may be desirable to space the outer surfaces 52 of the paddles 44 from the inner surface of the distal holding section 56 to reduce mechanical or frictional contact between the distal holding section 56 and the drug coating 54 to reduce drug loss during navigation to the treatment location and/or during actuation of the outer elongate shaft 14. In some examples, the connection members 46 may include a radially outward curve (not explicitly shown) that is configured to contact an inner surface of the distal holding section 56 when the drug delivery device 16 is disposed within the cavity 58 of the distal holding section 56. The curve or bend in the connection members 46 may deflect the paddles 44 radially inwards away from the inner surface of the distal holding section 56 to define a gap or clearance space between the outer surfaces 52 of the paddles 44 and the inner surface of the distal holding section 56. This may prevent or limit mechanical contact between the drug coating 54 and the inner surface of the distal holding section 56.

To deliver the therapeutic agent to the desired location, the system 10 may be navigated through the vasculature in the delivery configuration (with outer elongate shaft 14 distally advanced, to protect the drug coating 541 (e.g., drug load) while tracking and prevent drug loss due to friction) until the distal end region 34 is adjacent to the target location. In the delivery configuration, the outer elongate shaft 14 is positioned over the drug delivery device 16. In some examples, one or more radiopaque markers (not explicitly shown) may be included on the inner elongate shaft 12, the outer elongate shaft 14, and/or the drug delivery device 16 to facilitate placement of the drug delivery device 16 at the target location. Once the distal end region 34 and the drug delivery device 16 are adjacent to the target location, the operator may actuate the thumbwheel 68 of the handle 18 in a first direction to proximally retract the outer elongate shaft 14, as shown at arrow 86. The outer elongate shaft 14 may move relative to the handle 18, while the handle 18 and the inner elongate shaft 12 remain stationary or in a longitudinally fixed orientation. As the drug delivery device 16 is affixed to the inner elongate shaft 12, as the outer elongate shaft 14 is proximally retracted, the drug delivery device 16 may also remain in a longitudinally fixed orientation.

In FIG. 5, the outer elongate shaft 14 is in a partially proximally retracted configuration and only a distal portion of the drug delivery device 16 is outside of the distal holding section 56. As can be seen in FIG. 5, the distal holding section 56 maintains the drug delivery device 16 in a radially collapsed configuration. Further, proximal retraction of the outer elongate shaft 14 to position the distal holding section 56 proximal to the proximal end 38 or collar 50 of the drug delivery device 16 may allow the drug delivery device 16 to fully expand, as shown in FIG. 2. It is contemplated that in the radially expanded configuration, the drug delivery device 16 may have a maximum outer dimension that is greater than a maximum inner dimension of the distal holding section 56. In some cases, the lumen in which the drug delivery device 16 is deployed may limit the radial expansion of the drug delivery device 16 to a radial extent less than the maximum radial dimension. As the drug delivery device 16 expands, the outer surfaces 52 of the paddles 44 may contact the inner surface of the lumen in which the system 10 is deployed. The drug delivery device 16 may remain in the expanded configuration in apposition with the lumen wall for a length of time. In some cases, the drug delivery device 16 may contact the lumen wall for a time period in the range of about 30 to 60 seconds in the coronary arteries. However, the drug delivery device 16 may contact the lumen wall for less than 30 seconds or more than 60 seconds, as desired or necessary, depending on the treated anatomy and the drug dosage. As described above, the circumferential spacing of the struts 42 may allow blood or fluid perfusion through the drug delivery device 16 with no or minimal disruption as the drug delivery device 16 is in contact with the lumen wall. The drug coating 54 may be transferred from the outer surfaces 52 of the paddles 44 to the lumen wall. In some examples, the drug coating 54 may include a crystalline therapeutic agent, such as, but not limited to, crystalline paclitaxel or crystalline everolimus. The crystalline therapeutic agent may embed into the tissue and dissolve over a length of time. In some cases, the therapeutic agent may dissolve over a period of hours, days, weeks, etc. (for example, due to the gradual enthalpic dissolution of crystals transferred to the treated anatomy).

Once the drug coating 54 has been transferred to the lumen wall, the thumbwheel 68 may be actuated in a second direction, opposite the first direction, to distally advance the outer elongate shaft 14. The distal holding section 56 may exert a radially compressive force on the drug delivery device 16 to collapse the drug delivery device 16 as the distal holding section 56 is advanced over the drug delivery device 16. It is contemplated that the distal holding section 56 and/or the outer elongate shaft 14 may be formed from a material having a sufficient hoop strength to maintain the drug delivery device 16 in the radially collapsed configuration. The outer elongate shaft 14 may be distally advanced until the distal end 28 thereof contacts the cap 36 of the inner elongate shaft 12. The system 10 may then be withdrawn from the body. The drug coating 54 may remain in the body without the need to implant a drug coated medical device, such as, but not limited to, a drug coated stent. In some embodiments, the target location, may be prepped or pre-treated using balloon angioplasty, scoring balloons, atherectomy, or the like.

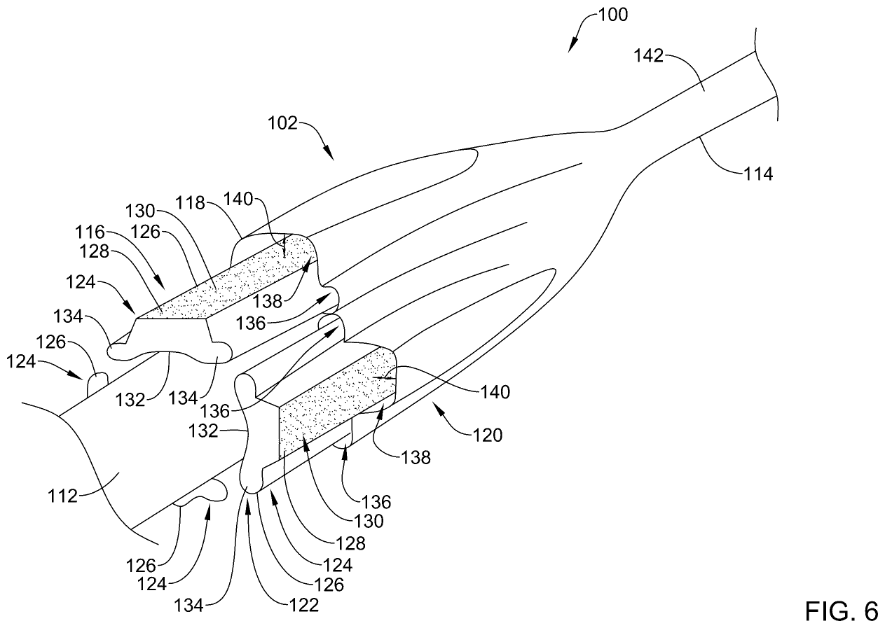

FIG. 6 is a schematic perspective view of a distal end region 102 of another illustrative drug transfer system 100. The illustrative system 100 may be similar in form and function to the system 10 described herein with an alternative expandable drug delivery device 116 and distal holding section 120. The system 100 may generally include an inner elongate shaft 112, an outer elongate shaft 114, an expandable drug delivery device 116, and a handle (not explicitly shown). The handle may be similar in form and function to the handle 18 described herein. Further, the inner elongate shaft 112 and the outer elongate shaft 114 may be coupled to the handle in a similar manner to that described with respect inner elongate shaft 12 and outer elongate shaft 14. The inner elongate shaft 112 may extend from a proximal end (not explicitly shown) coupled to the handle to a distal end (not explicitly shown). A lumen may extend from the proximal end to the distal end of the inner elongate shaft 112. While not explicitly shown, the lumen of the inner elongate shaft 112 may be configured to receive a guidewire for navigation and tracking. In some examples, the lumen may also be used to infuse diverse fluids, such as, but not limited to, saline solution, contrast media, or the like. The outer elongate shaft 114 may extend from a proximal end (not explicitly shown) movably coupled to the handle to a distal end 118. A lumen may extend from the proximal end to the distal end 118 of the outer elongate shaft 114. The inner elongate shaft 112 may be disposed within the lumen of the outer elongate shaft 114. In some embodiments, the outer elongate shaft 114 may be configured to be axially displaced (e.g., proximally or distally) along a longitudinal axis of the system 100 relative to the inner elongate shaft 112.

The cross-sectional dimensions of the inner elongate shaft 112 and/or the outer elongate shaft 114 may vary according to the desired application. Generally, the cross-sectional dimensions of the outer elongate shaft 114 may be sized smaller than the typical blood vessel in which the system 100 is to be used. The length of the outer elongate shaft 114 and/or the inner elongate shaft 112 may vary according to the location of the vascular passage where drug delivery is desired. In some instances, a 6 F or a 5 F catheter may be used as the outer elongate shaft 114. In some examples, intravascular ultrasound (IVUS) may be used for adequate sizing. Additionally, the inner elongate shaft 112 and/or the outer elongate shaft 114 or portions thereof may be selectively steerable. Mechanisms such as pull wires and/or other actuators may be used to selectively steer the inner elongate shaft 112 and/or the outer elongate shaft 114, if desired.

FIG. 6 illustrates the system with the outer elongate shaft 114 in a partially proximally retracted configuration. The partially proximally retracted configuration may be between a distally advanced delivery configuration of the outer elongate shaft 114 similar to that shown in FIG. 3 and a proximally retracted use configuration of the outer elongate shaft 114 similar to that shown in FIG. 2. While not explicitly shown, the inner elongate shaft 112 may include a nose cone, atraumatic bumper tip, or cap similar in form and function to the nose or cap 36 described herein positioned adjacent the distal end thereof. The cap may have an outer diameter that is greater than an outer diameter of the distal end of the inner elongate shaft 112, although this is not required. The expandable drug delivery device 116 may be coupled to an outer surface of the inner elongate shaft 112. The expandable drug delivery device 116 may be configured to transition from a radially collapsed delivery configuration to a radially expanded use configuration. The expandable drug delivery device 116 may extend from a proximal end (not explicitly shown) to a distal end 122.

It is contemplated that the expandable drug delivery device 116 may be formed from a number of different materials such as, but not limited to, metals, metal alloys, shape memory alloys and/or polymers, as desired, enabling the expandable drug delivery device 116 to be biased into a radially compressed configuration for delivery and a radially expanded configuration when the expandable drug delivery device 116 is in use. Depending on the material selected for construction, the expandable drug delivery device 116 may be self-expanding (i.e., configured to automatically expand when unconstrained). As used herein the term “self-expanding” refers to the tendency of the expandable drug delivery device 116 to return to a preprogrammed shape when unrestrained from an external biasing force (for example, but not limited to, the outer elongate shaft 114, etc.). For example, the expandable drug delivery device 116 may be heat set into a radially expanded configuration and compressed into a radially collapsed configuration for delivery within the outer elongate shaft 114. As the expandable drug delivery device 116 exits the outer elongate shaft 114, the expandable drug delivery device 116 may resume the radially expanded configuration. In some embodiments, the expandable drug delivery device 116 may be formed from nitinol. However, this is not required. Other shape memory alloys and/or polymers may be used, as desired. It is further contemplated that the drug delivery device 116 may be manually expanded and/or collapsed through actuation of a pull wire or other actuation mechanism.

The expandable drug delivery device 116 may include a plurality of generally longitudinally extending struts 124. While the illustrated embodiment depicts four struts 124, it is contemplated that the system 100 may include fewer than four or more than four struts, as desired. The struts 124 may be uniformly spaced about a circumference of the inner elongate shaft 112, although this is not required. In some cases, the struts 124 may be eccentrically or non-uniformly spaced about the circumference of the inner elongate shaft 112. It is contemplated that the struts 124 may be circumferentially spaced such that when the drug delivery device 116 is in a radially expanded configuration, the cross-section of the lumen in which drug delivery device 116 is deployed is not occluded. Said differently, when the drug delivery device 116 is in the radially expanded configuration blood or other fluid flows or perfuses with no or minimal disruption past the drug delivery device 116.

Each strut 124 may include a paddle 126 positioned adjacent to the distal end 122 of the expandable drug delivery device 116. The paddles 126 take any number of configurations desired. For example, the width of the paddles 126 may vary along a length thereof. In some cases, the width may taper or reduce from a distal end to a proximal end of the paddle 126. Other shapes and/or configurations may be used as desired. For example, the paddles 126 may have a shape (when viewed from an outer side) that is generally rectangular, square, triangular, circular, oblong, polygonal, eccentric, or the like.

A connection member similar in form and function to connection members 46 described herein may extend proximally from the proximal end of the paddle 126 to the proximal end of the expandable drug delivery device 116. The connection members may be secured to an outer surface of the inner elongate shaft 112 proximal to the distal end of the inner elongate shaft 112 via a collar or other suitable coupling mechanism. A suitable securing method(s) may be employed to couple the collar and the inner elongate shaft 112, including but not limited to adhesive bonding, thermal bonding (e.g., hot jaws, laser welding, etc.) or other bonding technique, as desired. In some examples, the connection members may have a width or cross-sectional dimension that is less than a width of the paddles 126. For example, the connection members may be sized and shaped to minimally impact the flow of blood when the expandable drug delivery device 116 is in the expanded configuration while securing the paddles 126 to the inner elongate shaft 112. In some examples, the connection members may be formed from a wire. Blood or fluid may flow through the spaces between the connection members. A distal end of the paddles 126 may be free from connection to the inner elongate shaft 112.

It is contemplated that the paddle 126 and the connection member of each individual strut 124 may be formed as a single monolithic structure that are each individually coupled to the inner elongate shaft 112. In other examples, the paddle 126 and the connection member each individual strut 124 may be formed as separate components that are coupled to one another. In yet other examples, the expandable drug delivery device 116 may be formed as a single monolithic component. For example, the struts 124 and the collar may be laser cut from a hypotube or may be 3-D printed.

The paddles 126 may include a radially outward surface 128 having a drug coating 130 disposed thereon. In some examples, the radially outward surface 128 may have a generally convex shape configured to conform to an inner surface of a lumen. However, this is not required. The radially outward surface 128 of the paddles 126 may take any shape desired. The drug coating 130 may be disposed along substantially the entire length and/or width of each paddle 126 or along one or more portions of the paddles 126. Some paddles 126 may be free from the drug coating 130. The drug coating 130 disposed on the paddles 126 may have an average thickness in the range of about 1 μm to about 50 μm, for example, although the coating thickness may depend on the morphology of the therapeutic agent (e.g., crystalline), the treated anatomy, and/or the required drug dose for therapeutic action. In some embodiments, a light binder (not explicitly shown) may hold the drug coating 130 to the paddles 126. It is contemplated that the therapeutic agent may be grown on the outer surfaces 128 of the paddles 126.

In some embodiments, a flexible sleeve (not explicitly shown) may extend circumferentially between the paddles 126. The drug coating 130 may be applied to the outer surface of the flexible sleeve. It is contemplated that the flexible sleeve, if so provided, may have length less than a length of the drug delivery device 116. This may allow the surface area available to apply the drug coating 130 to be increased (relative to only using the outer surface 128 of the paddles 126) while allowing blood or other fluid to perfuse through the spaces between the connection members.

The drug coating 130 may include one or more therapeutic agents such as, but not limited to, anti-thrombotic agents, anti-proliferative agents, anti-inflammatory agents, direct oral anticoagulants (DOACs), anti-migratory agents, agents affecting extracellular matrix production and organization, antineoplastic agents, anti-mitotic agents, anesthetic agents, anti-coagulants, vascular cell growth promoters, vascular cell growth inhibitors, cholesterol-lowering agents, vasodilating agents, agents that interfere with endogenous vasoactive mechanisms, proteomes, and cytokines. More specific drugs or therapeutic agents include paclitaxel, rapamycin, sirolimus, everolimus, tacrolimus, heparin, diclofenac, aspirin, Epo D, dexamethasone, estradiol, halofuginone, cilostazol, geldanamycin, apixaban, rivaroxaban, edoxaban, dabigatran, betrixaban, argatroban, ABT-578 (Zotarolimus, Abbott Laboratories), trapidil, liprostin, actinomycin D, Resten-NG, Ap-17, abciximab, clopidogrel, Ridogrel, beta-blockers, bARKct inhibitors, phospholamban inhibitors, and SERCA 2 gene/protein, resiquimod, imiquimod (as well as other imidazoquinoline immune response modifiers), human apolipoproteins (e.g., AI, AII, AIII, AIV, AV, etc.), vascular endothelial growth factors (e.g., VEGF-2), antibody CD-34, interleukin-8, as well as derivatives of the foregoing, among many others, and/or combinations thereof.

The paddles 126 may include a radially inward surface 132. The radially inward surface 132 may be curved or concave to generally conform to an outer surface of the inner elongate shaft 112. It is contemplated that the paddles 126 may be curved to reduce a maximum outer diameter of the drug delivery device 116 in the collapsed delivery configuration. The paddles 126 may further include one or more laterally extending protrusions or rails 134. The rails 134 may be disposed adjacent to the radially inward side of the paddles 126 such that a width of the paddles 126 adjacent to the radially inward surface 132 is greater than a width of the paddles 126 adjacent to the radially outward surface 128. The rails 134 may be configured to be received within mating channels of the distal holding section 120 to maintain the drug delivery device 116 in a desired position while the system 100 is navigated to the desired treatment location.

In general, the drug delivery device 116 may be delivered to a suitable target region via the system 100 while in the collapsed configuration with the outer elongate shaft 114 in a distally advanced or delivery configuration. Upon reaching the target region, the drug delivery device 116 may expand or be expanded into the expanded configuration. In the radially expanded configuration, the radially outward surfaces 128 of the paddles 126 may be configured to contact the lumen wall. The paddles 126 may be held in apposition with the lumen wall for a length of time sufficient to transfer the drug coating 130 from the paddles 126 to the lumen wall.

The outer elongate shaft 114 may be slidably disposed over the inner elongate shaft 112. The outer elongate shaft 114 may include a distal holding section 120 and a body portion 142. The body portion 142 may extend from the proximal end of the outer elongate shaft 114 to a proximal end of the distal holding section 120. The distal holding section 120 may extend distally from a distal end of the body portion 142 to the distal end 118 of the outer elongate shaft 114. In some embodiments, the body portion 142 and the distal holding section 120 may be formed as single monolithic structure. In other embodiments, the body portion 142 and the distal holding section 120 may be formed as separate components that are subsequently coupled. A suitable securing method(s) may be employed to couple the body portion 142 and the distal holding section 120, including but not limited to adhesive bonding, thermal bonding (e.g., hot jaws, laser welding, etc.) or other bonding technique, as desired. The body portion 142 and/or the distal holding section 120 may be manufactured using a number of differing techniques, including, but not limited to, extrusion, 3-D printing, injection molding, and the like.

The distal holding section 120 may have a cross-sectional dimension at the distal end 118 that is greater than a cross-sectional dimension of at least a portion of the outer elongate shaft 114 proximal to the distal holding section 120. In some examples, the cross-sectional dimension of the distal holding section 120 may increase from the proximal end of the distal holding section 120 to the distal end 118 of the outer elongate shaft 114. The distal holding section 120 may be configured to receive the drug delivery device 116 therein. For example, an entire length of the drug delivery device 116 may be contained within the distal holding section 120 during delivery to a target site. In other examples, the paddles 126 of the drug delivery device 116 may be housed within the distal holding section 120 while the connection members extend proximally into the body portion 142 of the outer elongate shaft 114. The distal holding section 120 may define a cavity for slidably receiving the drug delivery device 116. The distal holding section 120 may have a cross-sectional shape configured to accommodate the drug delivery device 116. The cavity may have recesses 138 sized and shaped to the receive the paddles 126 therein. It is contemplated that the number of recesses 138 may be the same as a number of paddles 126. The recesses 138 may include one or more longitudinally extending channels 136. The longitudinally extending channels may extend along a lateral side of the recesses 138. The channels 136 may be sized and shaped to receive the rails 134 of the paddles 126. The recesses 138 may have a height that is greater than a height of the paddles 126. Thus when the rails 134 are received within the channels 136, the radially outward surface 128 of the paddles 126 is spaced a distance 140 from an inner surface of the distal holding section 120. c

The cap of the inner elongate shaft 112 may be sized and shaped to mate with the distal end 118 of the outer elongate shaft 114 in the delivery configuration. This may enclose the drug delivery device 116 and prevent or reduce loss of the drug coating 130 as the drug delivery device 116 is navigated to the desired treatment location. The cap may taper or reduce in cross-sectional dimension in the distal direction, although this is not required. It is contemplated that the cap may have a cross-sectional shape similar to the cross-sectional shape of the distal holding section 120, although this is not required.

To deliver the therapeutic agent to the desired location, the system 100 may be navigated through the vasculature until the distal end region 102 is adjacent to the target location. In the delivery configuration, the outer elongate shaft 114 is positioned over the drug delivery device 116. In some examples, one or more radiopaque markers (not explicitly shown) may be included on the inner elongate shaft 112, the outer elongate shaft 114, and/or the drug delivery device 116 to facilitate placement of the drug delivery device 116 at the target location. Once the distal end region and the drug delivery device 116 is adjacent to the target location, the operator may actuate the thumbwheel of the handle in a first direction to proximally retract the outer elongate shaft 114. The outer elongate shaft 114 may move relative to the handle, while the handle and the inner elongate shaft 112 remain stationary or in a longitudinally fixed orientation. As the drug delivery device 116 is affixed to the inner elongate shaft 112, as the outer elongate shaft 114 is proximally retracted, the drug delivery device 116 may also remain in a longitudinally fixed orientation.

In FIG. 6, the outer elongate shaft 114 is in a partially proximally retracted configuration and only a distal portion of the drug delivery device 116 is outside of the distal holding section 120. As can be seen in FIG. 6, the distal holding section 120 maintains the drug delivery device 116 in a radially collapsed configuration. Further, proximal retraction of the outer elongate shaft 114 to position the distal holding section 120 proximal to the proximal end or collar of the drug delivery device 116 may allow the drug delivery device 116 to fully expand. It is contemplated that in the radially expanded configuration, the drug delivery device 116 may have a maximum outer dimension that is greater than a maximum inner dimension of the distal holding section 120. In some cases, the lumen in which the drug delivery device 116 is deployed may limit the radial expansion of the drug delivery device 116 to a radial extent less than the maximum radial dimension. As the drug delivery device 116 expands, the outer surfaces 128 of the paddles 126 may contact the inner surface of the lumen in which the system 100 is deployed. The drug delivery device 116 may remain in the expanded configuration in apposition with the lumen wall for a length of time. In some cases, the drug delivery device 116 may contact the lumen wall for a time period in the range of about 30 to 60 seconds. However, the drug delivery device 116 may contact the lumen wall for less than 30 seconds or more than 60 seconds, as desired or necessary. As described above, the circumferential spacing of the struts 124 may allow blood or fluid perfusion through the drug delivery device 116 with no or minimal disruption as the drug delivery device 116 is in contact with the lumen wall. The drug coating 130 may be transferred from the outer surfaces 128 of the paddles 126 to the lumen wall. In some examples, the drug coating 130 may include a crystalline therapeutic agent, such as, but not limited to, crystalline paclitaxel or crystalline everolimus. The crystalline therapeutic agent may embed into the tissue and dissolve over a length of time (e.g., gradual enthalpic dissolution of embedded crystals in the wall of the treated anatomy). In some cases, the therapeutic agent may dissolve over a period of hours, days, weeks, etc.

Once the drug coating 130 has been transferred to the lumen wall, the thumbwheel may be actuated in a second direction, opposite the first direction, to distally advance the outer elongate shaft 114. The distal holding section 120 may exert a radially compressive force on the drug delivery device 116 to collapse the drug delivery device 116 as the distal holding section 120 is advanced over the drug delivery device 116. It is contemplated that the distal holding section 120 and/or the outer elongate shaft 114 may be formed from a material having a sufficient hoop strength to maintain the drug delivery device 116 in the radially collapsed configuration. The outer elongate shaft 114 may be distally advanced until the distal end 118 thereof contacts the cap of the inner elongate shaft 112. The system 100 may then be withdrawn from the body. The drug coating 130 may remain in the body without the need to implant a drug coated medical device, such as, but not limited to, a drug coated stent. In some embodiments, the target location, may be prepped or pre-treated using balloon angioplasty, scoring balloons, atherectomy, or the like.

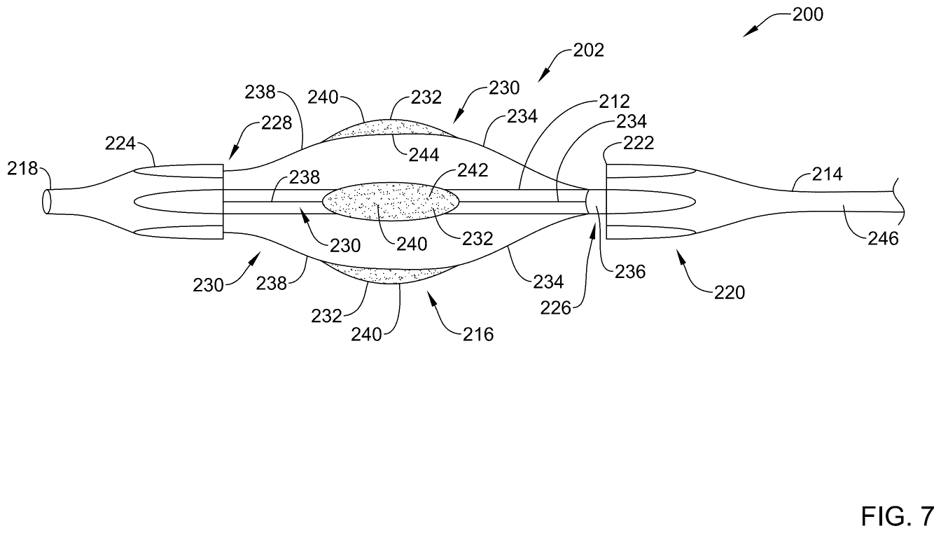

FIG. 7 is a schematic side view of a distal end region 202 of another illustrative drug transfer system 200 with the system 200 in an expanded, deployed configuration. The illustrative system 200 may be similar in form and function to the system 10 described herein with an alternative expandable drug delivery device 216 and distal holding section 220. The system 200 may generally include an inner elongate shaft 212, an outer elongate shaft 214, an expandable drug delivery device 216, and a handle (not explicitly shown). The handle may be similar in form and function to the handle 18 described herein. Further, the inner elongate shaft 212 and the outer elongate shaft 214 may be coupled to the handle in a similar manner to that described with respect inner elongate shaft 12 and outer elongate shaft 14. The inner elongate shaft 212 may extend from a proximal end (not explicitly shown) coupled to the handle to a distal end 218. A lumen may extend from the proximal end to the distal end 218 of the inner elongate shaft 212. While not explicitly shown, the lumen of the inner elongate shaft 212 may be configured to receive a guidewire for navigation and tracking. In some examples, the lumen may also be used to infuse diverse fluids, such as, but not limited to, saline solution, contrast media, or the like. The outer elongate shaft 214 may extend from a proximal end (not explicitly shown) movably coupled to the handle to a distal end 222. A lumen may extend from the proximal end to the distal end 222 of the outer elongate shaft 214. The inner elongate shaft 212 may be disposed within the lumen of the outer elongate shaft 214. In some embodiments, the outer elongate shaft 214 may be configured to be axially displaced (e.g., proximally or distally) along a longitudinal axis of the system 200 relative to the inner elongate shaft 212.

The cross-sectional dimensions of the inner elongate shaft 212 and/or the outer elongate shaft 214 may vary according to the desired application. Generally, the cross-sectional dimensions of the outer elongate shaft 214 may be sized smaller than the typical blood vessel in which the system 200 is to be used. The length of the outer elongate shaft 214 and/or the inner elongate shaft 212 may vary according to the location of the vascular passage where drug delivery is desired. In some instances, a 6 F or a 5 F catheter may be used as the outer elongate shaft 214. In some examples, intravascular ultrasound (IVUS) may be used for adequate sizing. Additionally, the inner elongate shaft 212 and/or the outer elongate shaft 214 or portions thereof may be selectively steerable. Mechanisms such as pull wires and/or other actuators may be used to selectively steer the inner elongate shaft 212 and/or the outer elongate shaft 214, if desired.

The inner elongate shaft 212 may include a nose cone, atraumatic bumper tip, or cap 224 similar in form and function to the nose or cap 36 described herein positioned adjacent the distal end thereof. The cap 224 may have an outer diameter that is greater than an outer diameter of the distal end 218, although this is not required. A proximal end 226 of the expandable drug delivery device 216 may be coupled to an outer surface of the inner elongate shaft 212 and a distal end 228 of the drug delivery device 216 may be coupled to the cap 224. The expandable drug delivery device 216 may be configured to transition from a radially collapsed delivery configuration (not explicitly shown) to a radially expanded use configuration, as shown in FIG. 7.

It is contemplated that the expandable drug delivery device 216 may be formed from a number of different materials such as, but not limited to, metals, metal alloys, shape memory alloys and/or polymers, as desired, enabling the expandable drug delivery device 216 to be biased into a radially compressed configuration for delivery and a radially expanded configuration when the expandable drug delivery device 216 is in use. Depending on the material selected for construction, the expandable drug delivery device 216 may be self-expanding (i.e., configured to automatically expand when unconstrained). As used herein the term “self-expanding” refers to the tendency of the expandable drug delivery device 216 to return to a preprogrammed shape when unrestrained from an external biasing force (for example, but not limited to, the outer elongate shaft 214, etc.). For example, the expandable drug delivery device 216 may be heat set into a radially expanded configuration and compressed into a radially collapsed configuration for delivery within the outer elongate shaft 214. As the expandable drug delivery device 216 exits the outer elongate shaft 214, the expandable drug delivery device 216 may resume the radially expanded configuration. In some embodiments, the expandable drug delivery device 216 may be formed from nitinol. However, this is not required. Other shape memory alloys and/or polymers may be used, as desired. It is further contemplated that the drug delivery device 216 may be manually expanded and/or collapsed through actuation of a pull wire or other actuation mechanism.

The expandable drug delivery device 216 may include a plurality of generally longitudinally extending struts 230. The system 200 may include any number of struts 230 desired, such as, one, two, three, four, or more. The struts 230 may be uniformly spaced about a circumference of the inner elongate shaft 212, although this is not required. In some cases, the struts 230 may be eccentrically or non-uniformly spaced about the circumference of the inner elongate shaft 212. It is contemplated that the struts 230 may be circumferentially spaced such that when the drug delivery device 216 is in a radially expanded configuration, the cross-section of the lumen in which drug delivery device 216 is deployed is not occluded. Said differently, when the drug delivery device 216 is in the radially expanded configuration blood or other fluid flows or perfuses with no or minimal disruption past the drug delivery device 216.

Each strut 230 may include a paddle 232 positioned between the proximal end 226 and the distal end 228 of the expandable drug delivery device 216. The paddles 232 take any number of configurations desired. For example, the width of the paddles 232 may vary along a length thereof. In some cases, the width may taper or reduce from a distal end to a proximal end of the paddle 232. Other shapes and/or configurations may be used as desired. For example, the paddles 232 may have a shape (when viewed from an outer side) that is generally rectangular, square, triangular, circular, oblong, polygonal, eccentric, or the like.

A proximal connection member 234 similar in form and function to connection members 46 described herein may extend proximally from the proximal end of the paddle 232 to the proximal end 226 of the expandable drug delivery device 216. The proximal connection members 234 may be secured to an outer surface of the inner elongate shaft 212 proximal to the distal end 218 thereof via a collar 236 or other suitable coupling mechanism. A suitable securing method(s) may be employed to couple the collar 236 and the inner elongate shaft 212, including but not limited to adhesive bonding, thermal bonding (e.g., hot jaws, laser welding, etc.) or other bonding technique, as desired. A distal connection member or tethering member 238 may extend distally from the distal end of the paddle 232 to the distal end 228 of the drug delivery device 216. The distal ends of the distal connection members 238 may be secured to the cap 224 or an outer surface of the inner elongate shaft 212. It is contemplated that tethering the paddles 232 to the cap 224 via the distal connection members 238 may restrict movement and allow for better control. A suitable securing method(s) may be employed to couple the distal connection members 238 and the inner elongate shaft 212 or cap 224 including but not limited to adhesive bonding, thermal bonding (e.g., hot jaws, laser welding, etc.) or other bonding technique, as desired.

In some examples, the proximal connection members 234 and/or distal connection members 238 may have a width or cross-sectional dimension that is less than a width of the paddles 232. For example, the proximal connection members 234 and/or distal connection members 238 may be sized and shaped to minimally impact the flow of blood when the expandable drug delivery device 216 is in the expanded configuration while securing the paddles 232 to the inner elongate shaft 212. In some examples, proximal connection members 234 and/or distal connection members 238 may be formed from a wire. Blood or fluid may flow through the spaces between the proximal connection members 234 and/or distal connection members 238.

It is contemplated that the paddle 232 and the proximal connection member 234 and/or distal connection member 238 of each individual strut 230 may be formed as a single monolithic structure that are each individually coupled to the inner elongate shaft 212. In other examples, the paddle 232 and the proximal connection member 234 and/or distal connection member 238 of each individual strut 230 may be formed as separate components that are coupled to one another. In yet other examples, the expandable drug delivery device 216 may be formed as a single monolithic component. For example, the struts 230 and the collar 236 may be laser cut from a hypotube or may be 3-D printed.

The paddles 232 may include a radially inward surface 244. The radially inward surface 244 may be curved or concave to generally conform to an outer surface of the inner elongate shaft 212. It is contemplated that the paddles 232 may be curved to reduce a maximum outer diameter of the drug delivery device 216 in the collapsed delivery configuration. The paddles 232 may include a radially outward surface 240 having a drug coating 242 disposed thereon. In some examples, the radially outward surface 240 may have a generally convex shape configured to conform to an inner surface of a lumen. However, this is not required. The radially outward surface 240 of the paddles 232 may take any shape desired. The drug coating 242 may be disposed along substantially the entire length and/or width of each paddle 232 or along one or more portions of the paddles 232. Some paddles 232 may be free from the drug coating 242. The drug coating 242 disposed on the paddles 232 may have an average thickness in the range of about 1 μm to about 50 μm, for example, although the coating thickness may vary depending on the morphology of the therapeutic agent (e.g., crystalline), the treated anatomy, and/or the required drug dosage. In some embodiments, a light binder (not explicitly shown) may hold the drug coating 242 to the paddles 232. It is contemplated that the therapeutic agent may be grown on the outer surfaces 240 of the paddles 232.

In some embodiments, a flexible sleeve (not explicitly shown) may extend circumferentially between the paddles 232. The drug coating 242 may be applied to the outer surface of the flexible sleeve. It is contemplated that the flexible sleeve, if so provided, may have length less than a length of the drug delivery device 216. This may allow the surface area available to apply the drug coating 242 to be increased (relative to only using the outer surface 240 of the paddles 232) while allowing blood or other fluid to perfuse through the spaces between the connection members.

The drug coating 242 may include one or more therapeutic agents such as, but not limited to, anti-thrombotic agents, anti-proliferative agents, anti-inflammatory agents, direct oral anticoagulants (DOACs), anti-migratory agents, agents affecting extracellular matrix production and organization, antineoplastic agents, anti-mitotic agents, anesthetic agents, anti-coagulants, vascular cell growth promoters, vascular cell growth inhibitors, cholesterol-lowering agents, vasodilating agents, agents that interfere with endogenous vasoactive mechanisms, proteomes, and cytokines. More specific drugs or therapeutic agents include paclitaxel, rapamycin, sirolimus, everolimus, tacrolimus, heparin, diclofenac, aspirin, Epo D, dexamethasone, estradiol, halofuginone, cilostazol, geldanamycin, apixaban, rivaroxaban, edoxaban, dabigatran, betrixaban, argatroban, ABT-578 (Zotarolimus, Abbott Laboratories), trapidil, liprostin, actinomycin D, Resten-NG, Ap-17, abciximab, clopidogrel, Ridogrel, beta-blockers, bARKct inhibitors, phospholamban inhibitors, and SERCA 2 gene/protein, resiquimod, imiquimod (as well as other imidazoquinoline immune response modifiers), human apolipoproteins (e.g., AI, AII, AIII, AIV, AV, etc.), vascular endothelial growth factors (e.g., VEGF-2), antibody CD-34, interleukin-8, as well as derivatives of the forgoing, among many others, and/or combinations thereof.

In general, the drug delivery device 216 may be delivered to a suitable target region via the system 200 while in the collapsed configuration with the outer elongate shaft 214 in a distally advanced or delivery configuration. Upon reaching the target region, the drug delivery device 216 may expand or be expanded into the expanded configuration. In the radially expanded configuration, the radially outward surfaces 240 of the paddles 232 may be configured to contact the lumen wall. The paddles 232 may be held in apposition with the lumen wall for a length of time sufficient to transfer the drug coating 242 from the paddles 232 to the lumen wall.

The outer elongate shaft 214 may be slidably disposed over the inner elongate shaft 212. The outer elongate shaft 214 may include a distal holding section 220 and a body portion 246. The body portion 246 may extend from the proximal end of the outer elongate shaft 214 to a proximal end of the distal holding section 220. The distal holding section 220 may extend distally from a distal end of the body portion 246 to the distal end 222 of the outer elongate shaft 214. In some embodiments, the body portion 246 and the distal holding section 220 may be formed as single monolithic structure. In other embodiments, the body portion 246 and the distal holding section 220 may be formed as separate components that are subsequently coupled. A suitable securing method(s) may be employed to couple the body portion 246 and the distal holding section 220, including but not limited to adhesive bonding, thermal bonding (e.g., hot jaws, laser welding, etc.) or other bonding technique, as desired. The body portion 246 and/or the distal holding section 220 may be manufactured using a number of differing techniques, including, but not limited to, extrusion, 3-D printing, injection molding, and the like.

The distal holding section 220 may have a cross-sectional dimension at the distal end 222 that is greater than a cross-sectional dimension of at least a portion of the outer elongate shaft 214 proximal to the distal holding section 220. In some examples, the cross-sectional dimension of the distal holding section 220 may increase from the proximal end of the distal holding section 220 to the distal end 222 of the outer elongate shaft 214. The distal holding section 220 may be configured to receive the drug delivery device 216 or portions of the drug delivery device therein. For example, an entire length of the drug delivery device 16 may be contained within the distal holding section 220 during delivery to a target site. Thus the distal holding section 220 may have a similar length to the drug delivery device. In other examples, the paddles 232 of the drug delivery device 216 may be housed within the distal holding section 220 while the proximal connection members 234 extend proximally into the body portion 246 of the outer elongate shaft 214. In some examples, the distal connection members 238 may not be received within the distal holding section 220 and thus the cap 224 may not contact the distal end 222 of the outer elongate shaft 214 in the delivery configuration.

The distal holding section 220 may define a cavity for slidably receiving the drug delivery device 216. The distal holding section 220 may have a cross-sectional shape configured to accommodate the drug delivery device 216. The cavity may have recesses sized and shaped to the receive the paddles 232 therein. It is contemplated that the number of recesses may be the same as a number of paddles 232. The distal holding section 220 and/or the paddles 232 may include features configured to space the radially outward surfaces 240 of the paddles 232 from the inner surface of the distal holding section 220 when the outer elongate shaft 214 is disposed over the drug delivery device 216 to reduce or prevent contact between the drug coating 242 and the inner surface of the distal holding section 220.

The cap 224 of the inner elongate shaft 212 may be sized and shaped to mate with the distal end 222 of the outer elongate shaft 214 in the delivery configuration, although this is not required. This may enclose the drug delivery device 216 and prevent or reduce loss of the drug coating 242 as the drug delivery device 216 is navigated to the desired treatment location. The cap 224 may taper or reduce in cross-sectional dimension in the distal direction, although this is not required. It is contemplated that the cap 224 may have a cross-sectional shape similar to the cross-sectional shape of the distal holding section 220, although this is not required.

To deliver the therapeutic agent to the desired location, the system 200 may be navigated through the vasculature until the distal end region 202 is adjacent to the target location. In the delivery configuration, the outer elongate shaft 214 is positioned over the drug delivery device 216. In some examples, one or more radiopaque markers (not explicitly shown) may be included on the inner elongate shaft 212, the outer elongate shaft 214, and/or the drug delivery device 216 to facilitate placement of the drug delivery device 216 at the target location. Once the distal end region and the drug delivery device 216 is adjacent to the target location, the operator may actuate the thumbwheel of the handle in a first direction to proximally retract the outer elongate shaft 214. The outer elongate shaft 214 may move relative to the handle, while the handle and the inner elongate shaft 212 remain stationary or in a longitudinally fixed orientation. As the drug delivery device 216 is affixed to the inner elongate shaft 212, as the outer elongate shaft 214 is proximally retracted, the drug delivery device 216 may also remain in a longitudinally fixed orientation.

In FIG. 7, the outer elongate shaft 214 is in a proximally retracted configuration and the drug delivery device 216 is in the radially expanded deployed configuration. Distal advancement of the outer elongate shaft 214 may capture the drug delivery device 216, or portions thereof, within the distal holding section 220 to radially collapse the drug delivery device 216. It is contemplated that in the radially expanded configuration, the drug delivery device 216 may have a maximum outer dimension that is greater than a maximum inner dimension of the distal holding section 220. In some cases, the lumen in which the drug delivery device 216 is deployed may limit the radial expansion of the drug delivery device 216 to a radial extent less than the maximum radial dimension. As the drug delivery device 216 expands, the outer surfaces 240 of the paddles 232 may contact the inner surface of the lumen in which the system 200 is deployed. The drug delivery device 216 may remain in the expanded configuration in apposition with the lumen wall for a length of time. In some cases, the drug delivery device 216 may contact the lumen wall for a time period in the range of about 30 to 60 seconds. However, the drug delivery device 216 may contact the lumen wall for less than 30 seconds or more than 60 seconds, as desired or necessary. As described above, the circumferential spacing of the struts 230 may allow blood or fluid perfusion through the drug delivery device 216 with no or minimal disruption as the drug delivery device 216 is in contact with the lumen wall. The drug coating 242 may be transferred from the outer surfaces 240 of the paddles 232 to the lumen wall. In some examples, the drug coating 242 may include a crystalline therapeutic agent, such as, but not limited to, crystalline paclitaxel or crystalline everolimus. The crystalline therapeutic agent may embed into the tissue and dissolve over a length of time (e.g., due to the gradual enthalpic dissolution of crystals transferred to the treated anatomy). In some cases, the therapeutic agent may dissolve over a period of hours, days, weeks, etc.

Once the drug coating 242 has been transferred to the lumen wall, the thumbwheel may be actuated in a second direction, opposite the first direction, to distally advance the outer elongate shaft 214. The distal holding section 220 may exert a radially compressive force on the drug delivery device 216 to collapse the drug delivery device 216 as the distal holding section 220 is advanced over the drug delivery device 216. It is contemplated that the distal holding section 220 and/or the outer elongate shaft 214 may be formed from a material having a sufficient hoop strength to maintain the drug delivery device 216 in the radially collapsed configuration. The outer elongate shaft 214 may be distally advanced until the distal end 222 thereof contacts the cap of the inner elongate shaft 212. The system 200 may then be withdrawn from the body. The drug coating 242 may remain in the body without the need to implant a drug coated medical device, such as, but not limited to, a drug coated stent. In some embodiments, the target location, may be prepped or pre-treated using balloon angioplasty, scoring balloons, atherectomy, or the like.

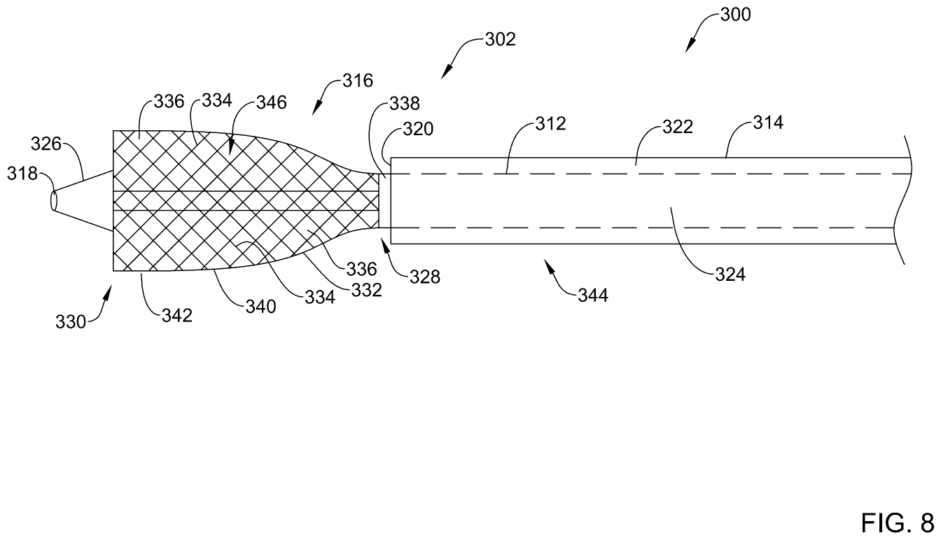

FIG. 8 is a schematic side view of a distal end region 302 of another illustrative drug transfer or delivery system 300 in an expanded, deployed configuration. The system 300 may generally include an inner elongate shaft 312, an outer elongate shaft 314, an expandable drug delivery device 316, and a handle (not explicitly shown). The handle may be similar in form and function to the handle 18 described herein. Further, the inner elongate shaft 312 and the outer elongate shaft 314 may be coupled to the handle in a similar manner to that described with respect inner elongate shaft 12 and outer elongate shaft 14. The inner elongate shaft 312 may extend from a proximal end (not explicitly shown) coupled to the handle to a distal end 318. A lumen 324 may extend from the proximal end to the distal end 318 of the inner elongate shaft 312. While not explicitly shown, the lumen 324 of the inner elongate shaft 312 may be configured to receive a guidewire for navigation and tracking. In some examples, the lumen 324 may also be used to infuse diverse fluids, such as, but not limited to, saline solution, contrast media, or the like. The outer elongate shaft 314 may extend from a proximal end (not explicitly shown) movably coupled to the handle to a distal end 320. A lumen 322 may extend from the proximal end to the distal end 320 of the outer elongate shaft 314. The inner elongate shaft 312 may be disposed within the lumen 322 of the outer elongate shaft 314. In some embodiments, the outer elongate shaft 314 may be configured to be axially displaced (e.g., proximally or distally) along a longitudinal axis of the system 300 relative to the inner elongate shaft 312.

The cross-sectional dimensions of the inner elongate shaft 312 and/or the outer elongate shaft 314 may vary according to the desired application. Generally, the cross-sectional dimensions of the outer elongate shaft 314 may be sized smaller than the typical blood vessel in which the system 300 is to be used. The length of the outer elongate shaft 314 and/or the inner elongate shaft 312 may vary according to the location of the vascular passage where drug delivery is desired. In some instances, a 6 F or a 5 F catheter may be used as the outer elongate shaft 314. In some examples, intravascular ultrasound (IVUS) may be used for adequate sizing. Additionally, the inner elongate shaft 312 and/or the outer elongate shaft 314 or portions thereof may be selectively steerable. Mechanisms such as pull wires and/or other actuators may be used to selectively steer the inner elongate shaft 312 and/or the outer elongate shaft 314, if desired.

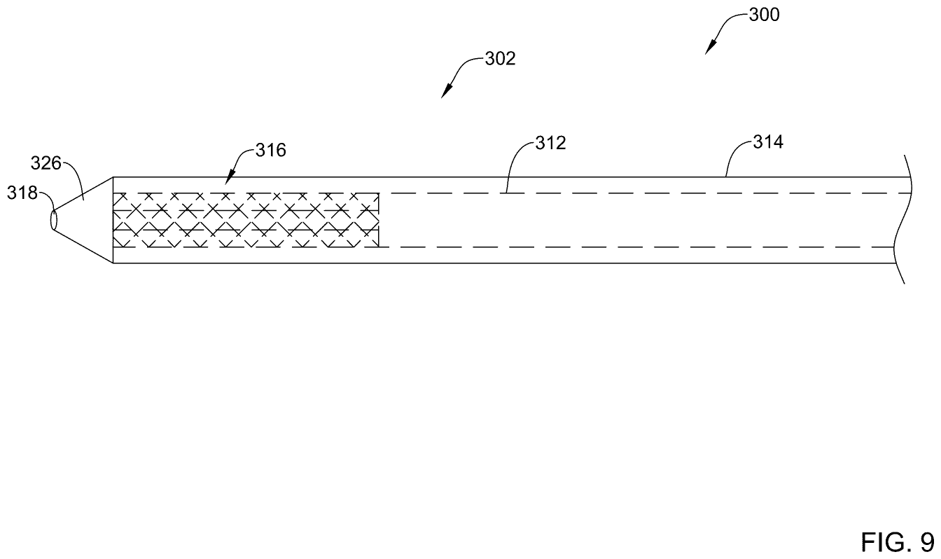

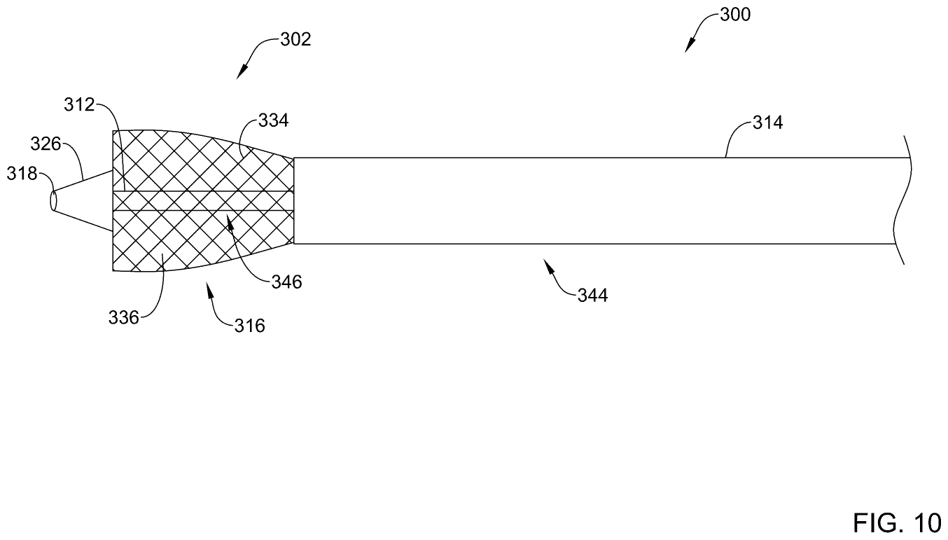

FIG. 9 is a side view of a distal end region 302 of the system 300 with the outer elongate shaft 314 in a distally advanced or delivery configuration. FIG. 10 is a side view of the distal end region 302 of the system 300 with the outer elongate shaft 314 in a partially proximally retracted configuration. The inner elongate shaft 312 may include a nose cone, atraumatic bumper tip, or cap 326 positioned adjacent the distal end 318 thereof. The cap 326 may have an outer diameter that is greater than an outer diameter of the distal end 318, although this is not required. The expandable drug delivery device 316 may be coupled to an outer surface of the inner elongate shaft 312. The expandable drug delivery device 316 may be configured to transition from a radially collapsed delivery configuration (see, for example, FIG. 9) to a radially expanded use configuration (see, for example, FIG. 8). The expandable drug delivery device 316 may extend from a proximal end 328 to a distal end 330.

In some embodiments, the inner elongate shaft 312 may include a reduced diameter region 346 adjacent to the drug delivery device 316. However, this is not required. When so provided, the reduced diameter region 346 may allow the drug delivery device 316 to be spaced from an inner surface of the outer elongate shaft 314 when the system 300 is in the delivery configuration. The spacing may reduce or prevent contact between an outer surface of the drug delivery device 316 and the inner surface of the outer elongate shaft 314 to reduce drug loss due to friction or rubbing during delivery of the system 300 or retraction of the outer elongate shaft 314.

It is contemplated that the expandable drug delivery device 316 may be formed from a number of different materials such as, but not limited to, metals, metal alloys, shape memory alloys and/or polymers, as desired, enabling the expandable drug delivery device 316 to be biased into a radially compressed configuration for delivery and a radially expanded configuration when the expandable drug delivery device 316 is in use. Depending on the material selected for construction, the expandable drug delivery device 316 may be self-expanding (i.e., configured to automatically expand when unconstrained). As used herein the term “self-expanding” refers to the tendency of the expandable drug delivery device 316 to return to a preprogrammed shape when unrestrained from an external biasing force (for example, but not limited to, the outer elongate shaft 314, etc.). For example, the expandable drug delivery device 316 may be heat set into a radially expanded configuration, as shown in FIG. 8, and compressed into a radially collapsed configuration for delivery within the outer elongate shaft 314. As the expandable drug delivery device 316 exits the outer elongate shaft 314, the expandable drug delivery device 316 may resume the radially expanded configuration. In some embodiments, the expandable drug delivery device 316 may be formed from nitinol. However, this is not required. Other shape memory alloys and/or polymers may be used, as desired. It is further contemplated that the drug delivery device 316 may be manually expanded and/or collapsed through actuation of a pull wire or other actuation mechanism.

In some instances, the drug delivery device 316 may be formed from an elongated tubular member 332. While the drug delivery device 316 is described as generally tubular, it is contemplated that the drug delivery device 316 may take any cross-sectional shape desired. As described above, the drug delivery device 316 may be expandable from a first radially collapsed configuration (FIG. 9) to a second radially expanded configuration (FIG. 8). In some cases, the drug delivery device 316 may be deployed to a configuration between the collapsed configuration and a fully expanded configuration. The drug delivery device 316 may be structured to apply a radially outward pressure to a target location to deliver a therapeutic agent.

The drug delivery device 316 may have a woven structure, fabricated from a number of filaments or struts 334 forming a tubular wall. In some embodiments, the drug delivery device 316 may be knitted or braided with a single filament or strut interwoven with itself and defining open cells 336 extending through the thickness of the tubular wall of the drug delivery device 316. In other embodiments, the drug delivery device 316 may be braided with several filaments or struts interwoven together and defining open cells 336 extending along a length and around the circumference of the tubular wall of the drug delivery device 316. The open cells 336 may each define an opening from an outer surface of the tubular wall to an inner surface of the tubular wall (e.g., through a thickness thereof) that is free from the filaments or struts 334. In another embodiment, the drug delivery device 316 may be knitted. In yet another embodiment, the drug delivery device 316 may be of a knotted type. In still another embodiment, the drug delivery device 316 may be a laser cut tubular member. A laser cut tubular member may have an open and/or closed cell geometry including one or more interconnected monolithic filaments or struts defining open cells 336 therebetween, with the open cells 336 extending along a length and around the circumference of the tubular wall. The open cells 336 may each define an opening from an outer surface of the tubular wall to an inner surface of the tubular wall (e.g., through a thickness thereof) that is free from the interconnected monolithic filaments or struts.

It is contemplated that the open cells 336 may be circumferentially and/or longitudinally spaced such that when the drug delivery device 316 is in a radially expanded configuration, the cross-section of the lumen in which drug delivery device 316 is deployed is not occluded. Said differently, when the drug delivery device 316 is in the radially expanded configuration blood or other fluid flows through the open cells 336 with no or minimal disruption past the drug delivery device 316.

The proximal end 328 of the drug delivery device 316 may be secured to an outer surface of the inner elongate shaft 312 proximal to the distal end 318 thereof via a collar or other suitable coupling mechanism 338 to allow the drug delivery device 316 to be re-captured. A suitable securing method(s) may be employed to couple the collar 338 and the inner elongate shaft 312, including but not limited to adhesive bonding, thermal bonding (e.g., hot jaws, laser welding, etc.) or other bonding technique, as desired. In some embodiments, the drug delivery device 316 and the collar 338 may be formed as separate components that are coupled to one another. In other embodiments, the drug delivery device 316 and the collar 338 may be formed as a single monolithic structure. While FIG. 8 illustrates the distal end 330 of the drug delivery device 316 as free from securement to the inner elongate shaft 312, in some embodiments, the distal end 330 of the drug delivery device 316 may be secured to the inner elongate shaft 312 and/or the cap 326.

The struts 334 may include a radially inward surface. The radially inward surface may be curved or concave to generally conform to an outer surface of the inner elongate shaft 312 in the collapsed configuration. It is contemplated that the struts 334 may be curved to reduce a maximum outer diameter of the drug delivery device 316 in the collapsed delivery configuration. The struts 334 may include a radially outward surface 340 having a drug coating 342 disposed thereon. In some examples, the radially outward surface 340 may have a generally convex shape configured to conform to an inner surface of a lumen. However, this is not required. The radially outward surface 340 of the struts 334 may take any shape desired. The drug coating 342 may be disposed along substantially the entire length and/or width of each strut 334 or along one or more portions of the struts 334. Some struts 334 may be free from the drug coating 342. The drug coating 342 disposed on the struts 334 may have an average thickness in the range of about 1 μm to about 50 μm, for example, although the coating thickness may depend on the morphology of the therapeutic agent (e.g., crystalline), the treated anatomy, and/or the required drug dose for therapeutic action. In some embodiments, a light binder (not explicitly shown) may hold the drug coating 342 to the struts 334. It is contemplated that the therapeutic agent may be grown on the outer surfaces 340 of the struts 334.

In some embodiments, a flexible sleeve (not explicitly shown) may extend circumferentially between the struts 334 covering at least some of the open cells 336. The drug coating 342 may be applied to the outer surface of the flexible sleeve. It is contemplated that the flexible sleeve, if so provided, may have length less than a length of the drug delivery device 316. For example, the flexible sleeve may not extend to the proximal end 328 of the drug delivery device 316. This may allow the surface area available to apply the drug coating 342 to be increased (relative to only using the outer surface 340 of the struts 334) while allowing blood or other fluid to perfuse through the open cells 336 adjacent to the proximal end 328 of the drug delivery device 316 and out a distal opening of the drug delivery device 316.

The drug coating 342 may include one or more therapeutic agents such as, but not limited to, anti-thrombotic agents, anti-proliferative agents, anti-inflammatory agents, direct oral anticoagulants (DOACs), anti-migratory agents, agents affecting extracellular matrix production and organization, antineoplastic agents, anti-mitotic agents, anesthetic agents, anti-coagulants, vascular cell growth promoters, vascular cell growth inhibitors, cholesterol-lowering agents, vasodilating agents, agents that interfere with endogenous vasoactive mechanisms, proteomes, and cytokines. More specific drugs or therapeutic agents include paclitaxel, rapamycin, sirolimus, everolimus, tacrolimus, heparin, diclofenac, aspirin, Epo D, dexamethasone, estradiol, halofuginone, cilostazol, geldanamycin, apixaban, rivaroxaban, edoxaban, dabigatran, betrixaban, argatroban, ABT-578 (Zotarolimus, Abbott Laboratories), trapidil, liprostin, actinomycin D, Resten-NG, Ap-17, abciximab, clopidogrel, Ridogrel, beta-blockers, bARKct inhibitors, phospholamban inhibitors, and SERCA 2 gene/protein, resiquimod, imiquimod (as well as other imidazoquinoline immune response modifiers), human apolipoproteins (e.g., AI, AII, AIII, AIV, AV, etc.), vascular endothelial growth factors (e.g., VEGF-2), antibody CD-34, interleukin-8, as well as derivatives of the foregoing, among many others, and/or combinations thereof.

In general, the drug delivery device 316 may be delivered to a suitable target region via the system 300 while in the collapsed configuration. Upon reaching the target region, the drug delivery device 316 may expand or be expanded into the expanded configuration. In the radially expanded configuration, the radially outward surfaces 340 of the struts 334 may be configured to contact the lumen wall. The struts 334 may be held in apposition with the lumen wall for a length of time sufficient to transfer the drug coating 342 from the struts 334 to the lumen wall.

The outer elongate shaft 314 may be slidably disposed over the inner elongate shaft 312. The distal end region 344 of the outer elongate shaft 314 may be configured to receive the drug delivery device 316 therein. For example, an entire length of the drug delivery device 316 may be contained within the distal end region 344 during delivery to a target site, although this is not required. In some cases, the drug delivery device 316 may be received within the lumen 322 of the outer elongate shaft 314. The distal end region 344 may have a cross-sectional shape configured to accommodate the drug delivery device 316.

The cap 326 of the inner elongate shaft 312 may be sized and shaped to mate with the distal end 320 of the outer elongate shaft 314 in the delivery configuration (see, for example, FIG. 9). This may enclose the drug delivery device 316 and prevent or reduce loss of the drug coating 342 as the drug delivery device 316 is navigated to the desired treatment location. Further, in the collapsed configuration, there may be enough clearance between the outer surface of the drug delivery device 316 and the inner surface of the outer elongate shaft 316 to prevent drug loss from friction between the movable outer elongate shaft 316 and the drug delivery device 316. The cap 326 may taper or reduce in cross-sectional dimension in the distal direction, although this is not required. It is contemplated that the cap 326 may have a cross-sectional shape similar to the cross-sectional shape of the distal end region 344, although this is not required.

To deliver the therapeutic agent to the desired location, the system 300 may be navigated through the vasculature until the distal end region 302 is adjacent to the target location. In the delivery configuration, the outer elongate shaft 314 is positioned over the drug delivery device 316. In some examples, one or more radiopaque markers (not explicitly shown) may be included on the inner elongate shaft 312, the outer elongate shaft 314, and/or the drug delivery device 316 to facilitate placement of the drug delivery device 316 at the target location. Once the distal end region 302 and the drug delivery device 316 is adjacent to the target location, the operator may actuate the thumbwheel of the handle in a first direction to proximally retract the outer elongate shaft 314. The outer elongate shaft 314 may move relative to the handle, while the handle and the inner elongate shaft 312 remain stationary or in a longitudinally fixed orientation. As the drug delivery device 316 is affixed to the inner elongate shaft 312, as the outer elongate shaft 314 is proximally retracted, the drug delivery device 316 may also remain in a longitudinally fixed orientation.

In FIG. 10, the outer elongate shaft 314 is in a partially proximally retracted configuration and only a distal portion of the drug delivery device 316 is outside of the distal end region 344. As can be seen in FIG. 10, the distal end region 344 of the outer elongate shaft 314 maintains the drug delivery device 316 in a radially collapsed configuration. Further proximal retraction of the outer elongate shaft 314 to position the distal end region 344 proximal to the proximal end or collar 338 of the drug delivery device 316 may allow the drug delivery device 316 to fully expand, as shown in FIG. 8. It is contemplated that in the radially expanded configuration, the drug delivery device 316 may have a maximum outer dimension that is greater than a maximum inner dimension of the distal end region 344. In some cases, the lumen in which the drug delivery device 316 is deployed may limit the radial expansion of the drug delivery device 316 to a radial extent less than the maximum radial dimension. As the drug delivery device 316 expands, the outer surfaces 340 of the struts 334 may contact the inner surface of the lumen in which the system 300 is deployed. The drug delivery device 316 may remain in the expanded configuration in apposition with the lumen wall for a length of time. In some cases, the drug delivery device 316 may contact the lumen wall for a time period in the range of about 30 to 60 seconds. However, the drug delivery device 316 may contact the lumen wall for less than 30 seconds or more than 60 seconds, as desired or necessary. As described above, the open cells 336 of the drug delivery device 316 may allow blood or fluid perfusion through the drug delivery device 316 with no or minimal disruption as the drug delivery device 316 is in contact with the lumen wall. The drug coating 342 may be transferred from the outer surfaces 340 of the struts 334 to the lumen wall. In some examples, the drug coating 342 may include a crystalline therapeutic agent, such as, but not limited to, crystalline paclitaxel or crystalline everolimus. The crystalline therapeutic agent may embed into the tissue and dissolve over a length of time (e.g., due to the gradual enthalpic dissolution of embedded crystals in the wall of the treated anatomy). In some cases, the therapeutic agent may dissolve over a period of hours, days, weeks, etc.

Once the drug coating 342 has been transferred to the lumen wall, the thumbwheel may be actuated in a second direction, opposite the first direction, to distally advance the outer elongate shaft 314. The distal end region 344 may exert a radially compressive force on the drug delivery device 316 to collapse the drug delivery device 316 as the distal end region 344 is advanced over the drug delivery device 316. It is contemplated that the distal end region 344 and/or the outer elongate shaft 314 may be formed from a material having a sufficient hoop strength to maintain the drug delivery device 316 in the radially collapsed configuration. The outer elongate shaft 314 may be distally advanced until the distal end 320 thereof contacts the cap 326 of the inner elongate shaft 312. The system 300 may then be withdrawn from the body. The drug coating 342 may remain in the body without the need to implant a drug coated medical device, such as, but not limited to, a drug coated stent. In some embodiments, the target location, may be prepped or pre-treated using balloon angioplasty, scoring balloons, atherectomy, or the like.

The materials that can be used for the various components of the drug delivery systems, and/or other devices disclosed herein may include those commonly associated with medical devices. For simplicity purposes, the following discussion makes reference to the drug delivery catheter system and its related components. However, this is not intended to limit the devices and methods described herein, as the discussion may be applied to other similar devices, tubular members and/or components of tubular members or devices disclosed herein.

The various components of the devices/systems disclosed herein may include a metal, metal alloy, polymer (some examples of which are disclosed herein), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In at least some embodiments, portions or all of the drug delivery system and its related components may be doped with, made of, or otherwise include a radiopaque material. Radiopaque materials are understood to be materials capable of producing a relatively bright image on a fluoroscopy screen or another imaging technique during a medical procedure. This relatively bright image aids the user of the drug delivery catheter system and its related components in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the drug delivery catheter system and its related components to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A drug delivery system, comprising:
an inner elongate shaft extending from a proximal end to a distal end and including a lumen extending from the proximal end to the distal end thereof;
an outer elongate shaft extending from a proximal end to a distal end and including a lumen extending from the proximal end to the distal end thereof, the outer elongate shaft disposed over and axially movable relative to the inner elongate shaft;
a self-expanding drug delivery device extending from a proximal end to a distal end, the self-expanding drug delivery device movable between a radially collapsed configuration and a radially expanded configuration, the self-expanding drug delivery device including a plurality of longitudinally extending struts; and
a drug coating disposed on at least a portion of a radially outward surface of the longitudinally extending struts.

2. The drug delivery system of claim 1, wherein the plurality of longitudinally extending struts each comprise a paddle and a proximally extending connection member, the proximally extending connection member having a width less than a width of the paddle.

3. The drug delivery system of claim 2, wherein the proximally extending connection member is coupled to the inner elongate shaft.

4. The drug delivery system of claim 2, wherein the plurality of longitudinally extending struts each comprise a distally extending connection member, the distally extending connection member having a width less than a width of the paddle.

5. The drug delivery system of claim 4, wherein the distally extending connection member is coupled to the inner elongate shaft adjacent the distal end thereof.

6. The drug delivery system of claim 1, wherein the outer elongate shaft comprises a distal holding section disposed adjacent the distal end of the outer elongate shaft, the distal holding section configured to maintain the self-expanding drug delivery device in a radially collapsed configuration.

7. The drug delivery system of claim 6, wherein the distal holding section has a cross-sectional dimension greater than a cross-sectional dimension of the outer elongate shaft proximal to the distal holding section.

8. The drug delivery system of claim 6, wherein the distal holding section defines a cavity having a plurality of recesses sized and shaped to receive a portion of the longitudinally extending struts.

9. The drug delivery system of claim 1, wherein the plurality of longitudinally extending struts are spaced about a circumference of the inner elongate shaft.

10. The drug delivery system of claim 1, wherein proximal retraction of the outer elongate shaft is configured to allow the self-expanding drug delivery device to move from the radially collapsed configuration to the radially expanded configuration.

11. The drug delivery system of claim 1, wherein the inner elongate shaft further comprises a cap disposed adjacent to the distal end thereof.

12. The drug delivery system of claim 11, wherein the cap is configured to be disposed adjacent to the distal end of the outer elongate shaft when the self-expanding drug delivery device is in the radially collapsed configuration.

13. The drug delivery system of claim 1, further comprising a handle coupled to the proximal end of the inner elongate shaft and the proximal end of the outer elongate shaft, the outer elongate shaft configured to be axially displaced relative to the handle.

14. A drug delivery system, comprising:
an inner elongate shaft extending from a proximal end to a distal end and including a lumen extending from the proximal end to the distal end thereof;
an outer elongate shaft extending from a proximal end to a distal end and including a lumen extending from the proximal end to the distal end thereof, the outer elongate shaft disposed over and axially movable relative to the inner elongate shaft;
a self-expanding drug delivery device extending from a proximal end to a distal end, the self-expanding drug delivery device movable between a radially collapsed configuration and a radially expanded configuration, the self-expanding drug delivery device including a plurality of longitudinally extending struts;
a distal holding section disposed adjacent the distal end of the outer elongate shaft, the distal holding section configured to constrain the self-expanding drug delivery device in the radially collapsed configuration when the self-expanding drug delivery device is disposed within a cavity of the distal holding section; and
a drug coating disposed on at least a portion of a radially outward surface of the longitudinally extending struts;
wherein when the self-expanding drug delivery device is disposed within the cavity of the distal holding section, the radially outward surface of the longitudinally extending struts are spaced a distance from an inner surface of the distal holding section.

15. The drug delivery system of claim 14, wherein the cavity of the distal holding section having a plurality of recesses sized and shaped to receive a portion of the longitudinally extending struts.

16. The drug delivery system of claim 15, wherein the plurality of recesses each include one or more longitudinally extending channels, the one or more longitudinally extending channels extending along a lateral side of said recess.

17. The drug delivery system of claim 16, wherein the plurality of longitudinally extending struts comprise one or more rails configured to be received within the one or more longitudinally extending channels.

18. The drug delivery system of claim 14, further comprising a handle coupled to the proximal end of the inner elongate shaft and the proximal end of the outer elongate shaft, the outer elongate shaft configured to be axially displaced relative to the handle.

19. The drug delivery system of claim 14, wherein proximal retraction of the outer elongate shaft is configured to allow the self-expanding drug delivery device to move from the radially collapsed configuration to the radially expanded configuration.

20. A drug delivery system, comprising:
an inner elongate shaft extending from a proximal end to a distal end and including a lumen extending from the proximal end to the distal end thereof;
an outer elongate shaft extending from a proximal end to a distal end and including a lumen extending from the proximal end to the distal end thereof, the outer elongate shaft disposed over and axially movable relative to the inner elongate shaft;
a self-expanding drug delivery device extending from a proximal end to a distal end, the self-expanding drug delivery device movable between a radially collapsed configuration and a radially expanded configuration, the self-expanding drug delivery device including one or more interwoven struts defining a plurality of open cells; and
a drug coating disposed on at least a portion of a radially outward surface of the interwoven struts.
